(19) United States
(12) Patent Application Publication
(10) Pub. No.: US 2025/0352235 A1
(43) Pub. Date: Nov. 20, 2025

(54) ULTRASONIC ROBOTICALLY DRIVEN SURGICAL INSTRUMENT

(71) Applicant: CMR Surgical Limited, Cambridge (GB)

(72) Inventors: André FICHTNER, Wildberg (DE); Christopher Norman GRAHAM, Devon (GB); Lothar MITZLAFF, Lagos (PT); Paul Lewis RICKETTS, Devon (GB); Michael Peter THOß, Nagold (DE)

(73) Assignee: CMR Surgical Limited, Cambridge (GB)

(21) Appl. No.: 19/209,199

(22) Filed: May 15, 2025

(30) Foreign Application Priority Data
May 15, 2024 (GB) 2406882.7
May 15, 2024 (GB) 2406883.5
May 15, 2024 (GB) 2406885.0

(51) Int. Cl. A61B 17/32; A61B 17/00; A61B 34/30

(52) U.S. Cl. CPC A61B 17/320092; A61B 34/30; A61B 2017/00199; A61B 2017/00477; A61B 2017/320071

(57) ABSTRACT

A robotic surgical instrument comprising: an articulated end effector; an instrument interface configured to engage with and being driven by a corresponding robot arm interface of a surgical robot arm; a casing housing a drive mechanism connected to the instrument interface, the drive mechanism comprising a drive gear; and a shaft pivotally connected to the casing at a proximal end and connected to the articulated end effector at a distal end, the shaft comprising a driveable shaft member configured to drive articulation of the articulated end effector, and a shaft gear attached to the driveable shaft member, the shaft gear meshing with the drive gear such that rotation of the drive gear drives the shaft gear to rotate which drives the driveable shaft member which drives articulation of the articulated end effector, wherein the shaft gear is pivotable with respect to the drive gear when the shaft gear and drive gear are meshed together.

### RELATED APPLICATIONS

This application claims priority to G.B. Application Nos. 2406883.5, 2406882.7, and 2406885.0, filed May 15, 2024, each of which is hereby incorporated herein by reference in its entirety.

### BACKGROUND

It is known to use robots for assisting and performing surgery. FIG. 1 illustrates a typical surgical robotic system. A surgical robot 100 consists of a base 102, an arm 104 and an instrument 106. The base supports the robot, and may itself be attached rigidly to, for example, the operating theatre floor, the operating theatre ceiling or a cart. The arm extends between the base and the instrument. The arm is articulated by means of multiple flexible joints 108 along its length, which are used to locate the surgical instrument in a desired location relative to the patient. The surgical instrument is attached to the distal end of the robot arm. The surgical instrument penetrates the body of the patient at a port so as to access the surgical site. The surgical instrument comprises a shaft connected to a distal end effector 110 by a jointed articulation 111. The end effector engages in a surgical procedure at the surgical site.

A surgeon controls the surgical robot 100 via a remote surgeon console 112. The surgeon console comprises one or more surgeon input devices 114. These may take the form of a hand controller or foot pedal. The surgeon console also comprises a display 116.

A control system 118 connects the surgeon console 112 to the surgical robot 100. The control system receives sensory inputs from the robot 100 and command inputs from the surgeon input device(s) 114. The control system uses these inputs to calculate control signals to move the joints of the robot arm 104 and instrument 106. The control system sends these control signals to the robot, where the corresponding joints are driven accordingly.

Different types of surgical instrument are used for different purposes at the surgical site. For example, the end effector may be one of a scalpel, gripping jaws, scissors, and a needle holder. Surgical instruments are either “cold” or “hot”. Cold instruments are not energised, whereas hot instruments are energised and apply heat to tissue at the surgical site. This is useful for cutting operations, particularly on dense or fibrous tissue which is difficult to penetrate with a cold instrument. It is also useful for sealing operations, for example to seal a blood vessel prior to cutting through the vessel between the sealed sections. Hot instruments are typically electrosurgical instruments. An electrosurgical power cable is fed through the shaft of the instrument to apply a high frequency electric current to electrodes located on the end effector. The end effector is thus live when the instrument is energised. In monopolar electrosurgical instruments, current passes from the end effector to tissue at the surgical site and then returns via a separate return electrode placed on the patient. In bipolar electrosurgical instruments, current passes from an electrode of the end effector to tissue at the surgical site and then returns via a return electrode of the bipolar instrument, for example located elsewhere on the end effector.

Although effective, electrosurgical instruments risk burns caused by unintended application of energy to tissue, for example through capacitive coupling. Instead of using electrosurgical instruments, energy may instead be provided via ultrasonic instruments. Ultrasonic instruments heat up tissue at the surgical site via rapid oscillation of the end effector. A piezoelectric transducer is used to convert alternating current into high frequency small amplitude oscillatory movements of a waveguide, those movements being transferred to the end effector. Ultrasonic instruments are hot instruments useful for the same cutting and sealing operations as electrosurgical instruments. They are safer than electrosurgical instruments because they can be used to apply heat without the risk of burns. Additionally, they are able to cut through tissue without the tissue needing to be clamped between jaws, thus are useful as a general dissector for various types of tissue.

### SUMMARY OF THE INVENTION

According to an aspect of the invention, there is provided a robotic surgical instrument comprising: an articulated end effector; an instrument interface for engaging with and being driven by a corresponding robot arm interface of a surgical robot arm; a casing housing a drive mechanism connected to the instrument interface, the drive mechanism comprising a drive gear; and a shaft pivotally connected to the casing at a proximal end and connected to the articulated end effector at a distal end, the shaft comprising a driveable shaft member for driving articulation of the articulated end effector, and a shaft gear attached to the driveable shaft member, the shaft gear meshing with the drive gear such that rotation of the drive gear drives the shaft gear to rotate which drives the driveable shaft member which drives articulation of the articulated end effector, wherein the shaft gear is pivotable with respect to the drive gear when the shaft gear and drive gear are meshed together.

The teeth of the shaft gear may have rounded ends such that when meshed with the teeth of the drive gear, each tooth of the shaft gear extends only partway down the valley between adjacent teeth of the drive gear.

The teeth of the drive gear may have rounded ends such that when meshed with the teeth of the shaft gear, each tooth of the drive gear extends only partway down the valley between adjacent teeth of the shaft gear.

The shaft may comprise a protrusion rigidly attached to the shaft, wherein the protrusion extends in a direction perpendicular to the longitudinal axis of the driveable shaft member.

The shaft gear may comprise a ring surrounding the driveable shaft member, the teeth of the shaft gear extending from the ring, and the protrusion extending from the ring in a direction opposing the teeth of the shaft gear.

The casing may comprise a notch which constrains the protrusion when the longitudinal axis of the shaft is angled relative to the longitudinal axis of the instrument interface.

The articulated end effector may comprise a pair of opposable end effector elements, the opening angle between the end effector elements driveable by the driveable shaft member, wherein when the protrusion is constrained within the notch, the end effector elements are in an open configuration.

When the protrusion is constrained within the notch, the opening angle between the end effector elements may be greater than 200.

The drive gear may be a part gear.

The teeth of the drive gear may extend less than 90° around its centre.

The shaft gear may be a part gear.

The teeth of the shaft gear may extend less than 90° around the driveable shaft member.

The driveable shaft member may be a rotatable shaft member, and the drive mechanism comprise: a transmission structure configured to transfer drive by moving linearly; and a drive assembly for converting linear motion of the transmission structure to rotational motion for driving the rotatable shaft member, the drive assembly comprising a helical drive driveable by the transmission structure, the drive gear rigidly attached to the helical drive.

The longitudinal axis of the helical drive may be offset from the longitudinal axis of the rotatable shaft member.

The longitudinal axis of the helical drive may be parallel to the longitudinal axis of the rotatable shaft member.

The helical drive, rotatable shaft member, drive gear and shaft gear may be located in the same plane perpendicular to the longitudinal axes of the helical drive and rotatable shaft member.

The teeth of the drive gear may extend in a direction perpendicular to the longitudinal axis of the helical drive.

The teeth of the shaft gear may extend in a direction perpendicular to the longitudinal axis of the driveable shaft member.

The teeth of the drive gear may mesh with the teeth of the shaft gear in a direction perpendicular to the longitudinal axis of the helical drive.

The drive gear may mesh with the shaft gear such that rotation of the helical drive in one rotational direction is converted to rotation of the rotatable shaft member in the opposing rotational direction.

According to an aspect of the invention, there is provided a method of assembling a robotic surgical instrument comprising a first portion and a second portion, the first portion comprising a rod connected to a first end effector element at a distal end and an instrument body at a proximal end, the second portion comprising an instrument interface for engaging with and being driven by a corresponding robot arm interface of a surgical robot arm, a casing housing a drive mechanism connecting the instrument interface to a shaft, the shaft connecting the drive mechanism to a second end effector element, the shaft comprising a driveable shaft member for driving articulation of the second end effector element, a protrusion attached to the driveable shaft member and extending in a direction perpendicular to the longitudinal axis of the driveable shaft member, the protrusion retainable in a notch in the casing, the method comprising: inserting the rod of the first portion into the driveable shaft member of the second portion; rotating the rod so as to rotate the driveable shaft member until the protrusion is aligned with the notch of the casing and the second end effector element adopts an open configuration; pivoting the driveable shaft member relative to the casing such that the longitudinal axis of the driveable shaft member is angled relative to the longitudinal axis of the instrument interface so as to cause the protrusion to be retained in the notch; fully inserting the rod into the driveable shaft member; and pivoting the driveable shaft member relative to the casing such that the longitudinal axis of the driveable shaft member is parallel to the longitudinal axis of the instrument interface and the instrument body is retained in the casing of the second portion.

### BRIEF DESCRIPTION OF THE FIGURES

The present invention will now be described by way of example with reference to the accompanying drawings. In the drawings:

FIG. 1 illustrates a surgical robot system for performing a surgical procedure;

FIG. 2 illustrates a surgical robot;

FIG. 3 illustrates an ultrasonic instrument;

FIG. 4 illustrates two portions of an ultrasonic instrument prior to assembly;

FIG. 5 illustrates part of an assembled ultrasonic instrument;

FIG. 6 illustrates the distal end of an assembled ultrasonic instrument;

FIG. 7 illustrates the proximal end of the second portion of the ultrasonic instrument;

FIG. 8 illustrates the drive assembly of the second portion of the ultrasonic instrument;

FIG. 9 illustrates assembly of the first and second portions to form the assembled ultrasonic instrument;

FIG. 10 illustrates the drive and shaft gears in the assembled ultrasonic instrument;

FIG. 11 illustrates the meshing of the teeth of the drive and shaft gears;

FIGS. 12a and 12b illustrate alignment features utilised when assembling the first and second portions of the ultrasonic instrument;

FIG. 13 illustrates the transmission structure in isolation;

FIG. 14 illustrates the instrument interface;

FIG. 15 illustrates the proximal end of the transmission structure;

FIG. 16 illustrates the underside of the transmission structure shown in FIG. 13 in isolation;

FIG. 17 illustrates the slider projection of the transmission structure when incorporated into the casing of the instrument interface;

FIG. 18 illustrates a moveable latch of the instrument with the instrument casing omitted;

FIG. 19 illustrates the moveable latch of the instrument with the instrument casing in place;

FIG. 20 illustrates the moveable latch during assembly of the first and second portions of the instrument;

FIG. 21 illustrates the instrument body of the first portion of the instrument;

FIGS. 22 and 23 illustrate a casing of the second portion of the instrument viewed from different angles; and

FIG. 24 illustrates a cross section of the instrument engaged with the robot arm.

### DETAILED DESCRIPTION

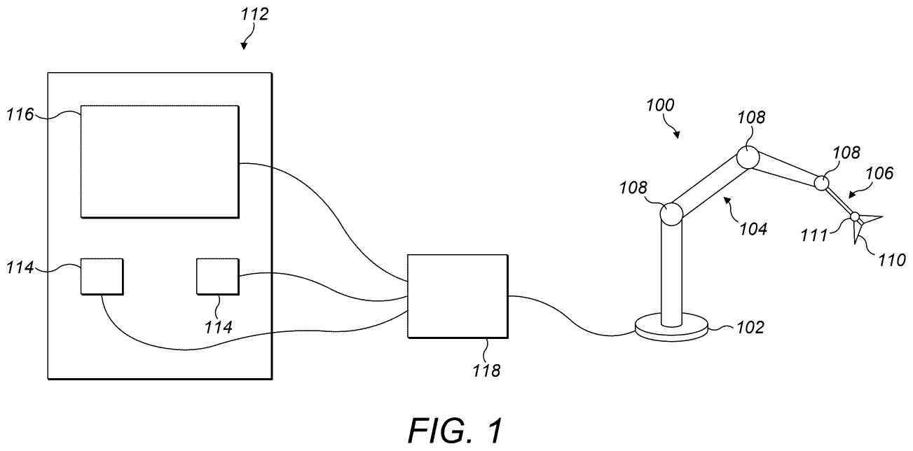

The following describes an ultrasonic robotic surgical instrument suitable for being mounted to and driven by a surgical robot arm. The surgical robot arm may be controlled by a remote surgeon console. The ultrasonic robotic surgical instrument, the surgical robot arm and the surgeon console form part of a surgical robotic system of the type described with reference to FIG. 1.

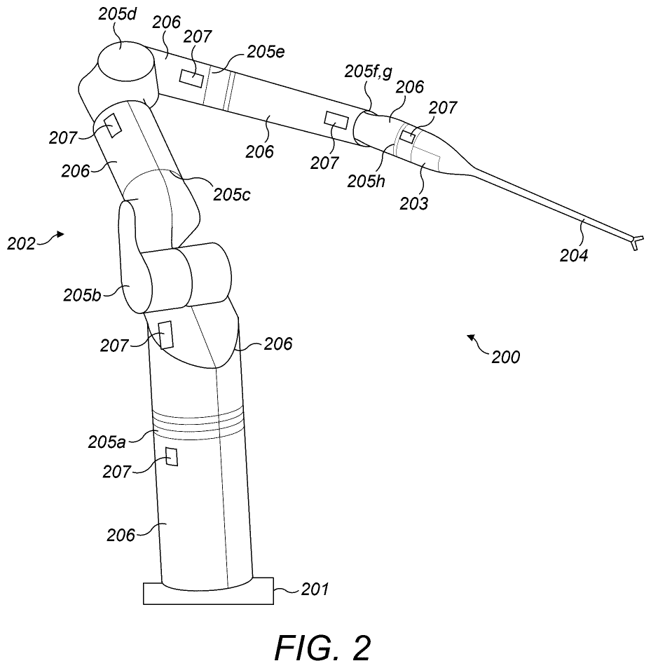

FIG. 2 illustrates an example robot 200. The robot comprises a base 201 which is fixed in place when a surgical procedure is being performed. Suitably, the base 201 is mounted to a chassis. That chassis may be a cart, for example a bedside cart for mounting the robot at bed height. Alternatively, the chassis may be a ceiling mounted device, or a bed mounted device.

A robot arm 202 extends from the base 201 of the robot to a terminal link 203 to which a surgical instrument 204 can be attached. The arm is flexible. It is articulated by means of multiple flexible joints 205 along its length. In between the joints are rigid arm links 206. The arm in FIG. 2 has eight joints. The joints include one or more roll joints (which have an axis of rotation along the longitudinal direction of the arm members on either side of the joint), one or more pitch joints (which have an axis of rotation transverse to the longitudinal direction of the preceding arm member), and one or more yaw joints (which also have an axis of rotation transverse to the longitudinal direction of the preceding arm member and also transverse to the rotation axis of a co-located pitch joint). In the example of FIG. 2: joints 205a, 205c, 205e and 205h are roll joints; joints 205b, 205d and 205f are pitch joints; and joint 205g is a yaw joint. Pitch joint 205f and yaw joint 205g have intersecting axes of rotation.

The order of the joints from the base 201 to the terminal link 203 of the robot arm is thus: roll, pitch, roll, pitch, roll, pitch, yaw, roll. However, the arm could be jointed differently. For example, the arm may have fewer than eight or more than eight joints. The arm may include joints that permit motion other than rotation between respective sides of the joint, for example a telescopic joint. The robot comprises a set of drivers 207. Each driver 207 has a motor which drives one or more of the joints 205. The terminal link 203 of the robot arm comprises a robot arm drive assembly for interfacing and driving a surgical instrument. The robot arm drive assembly comprises drive assembly interface elements which engage with corresponding instrument interface elements of an instrument interface of the surgical instrument. The drive assembly interface elements are driven by drivers 207. As the drive assembly interface elements move they move the instrument interface elements they are engaged with, thereby transferring drive from the drive assembly of the robot arm to the instrument interface of the instrument.

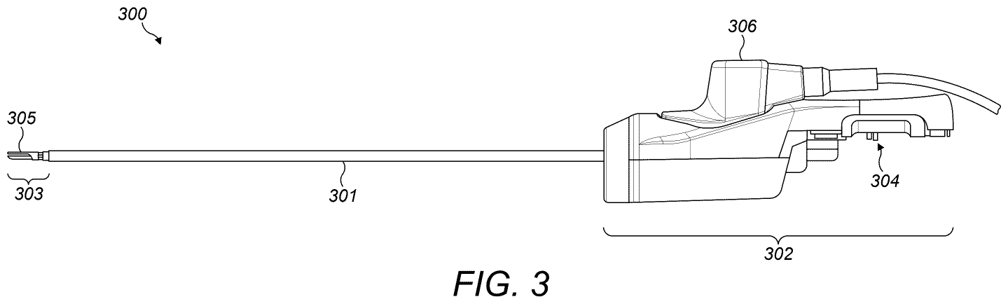

FIG. 3 illustrates an ultrasonic instrument 300. The ultrasonic instrument has an elongate profile, with a shaft 301 spanning between its proximal end 302 which is attached to the robot arm and its distal end 303 which accesses the surgical site within the patient body. At the proximal end of the ultrasonic instrument, an instrument interface 304 engages with the robot arm interface at the distal end of the robot arm. Drive is transferred from the robot arm to the ultrasonic surgical instrument at this interface. That drive is transferred from the instrument interface to the shaft of the instrument. At the distal end of the surgical instrument, the distal end of the shaft is connected to an end effector 305. The shaft transfers drive to the end effector 305 for articulating the end effector. The proximal end of the ultrasonic instrument comprises a transducer 306 which causes oscillation of a waveguide in the shaft. That oscillation is transferred to the end effector 305 for heating tissue. Thus, the end effector is able to both mechanically articulate to manipulate tissue at the surgical site and heat the tissue at the surgical site.

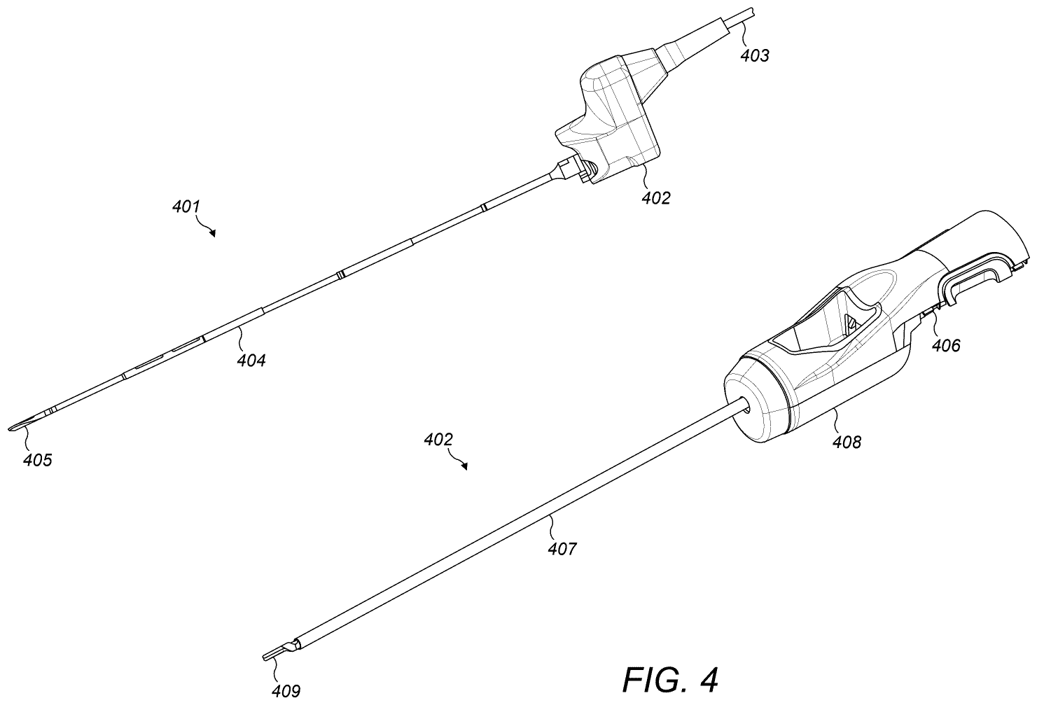

The ultrasonic instrument may comprise two separate portions 401, 402, which are assembled together prior to attaching the ultrasonic instrument to the robot arm. These portions are illustrated in FIG. 4. The first portion 401 comprises those features of the ultrasonic instrument which provide the application of energy to the end effector via vibration. The second portion 402 comprises those features of the ultrasonic instrument which provide the articulation of the end effector that is driven by the robot arm. The first portion may be reused from operation to operation. In this case, it is cleaned between operations. The second portion may be single use. In other words, the second portion may be disposable. Alternatively, the second portion may be reused from operation to operation, being cleaned and optionally refurbished between operations. The refurbishment may comprise, for example, replacing the protective pad (described in more detail below).

The first portion 401 comprises an instrument body 402. The instrument body 402 houses a piezo-electric transducer for converting alternating current to high frequency vibrations. In the example shown in FIG. 4, the transducer is powered by power cable 403. Power cable 403 may be powered by a generator which is external to the robot arm. Instead, the power cable may be fed through the robot arm and to the transducer via the robot arm/instrument interface. The first portion also comprises a waveguide 404. The waveguide 404 has an elongate profile. The waveguide may be straight. The waveguide may be rigid. The waveguide may be stiff. The waveguide is connected to the transducer at its proximal end, and connected to an end effector element 405 at its distal end. The end effector element 405 may take any suitable form. In the example shown in FIG. 4, the end effector element 405 is a jaw. This jaw is the lower jaw of an end effector which further comprises an upper jaw when the first and second portions of the ultrasonic instrument are assembled together.

Typically, ultrasonic devices vibrate in an axial direction parallel to the longitudinal axis of the waveguide. This allows perforation of tissue planes using the tip of the waveguide. However, the waveguide tip may unintentionally damage surrounding tissue. The waveguide described herein is preferably a rotating or torsional waveguide. However, the waveguide described herein may alternatively be a linear waveguide that vibrates in an axial direction parallel to the longitudinal axis of the waveguide. A torsional waveguide vibrates in a rotational manner about its longitudinal axis. This achieves the same heating effect on the tissue it contacts as is achieved by a waveguide which vibrates axially, however, it reduces the chance of unintentional perforations of tissue caused by the waveguide tip.

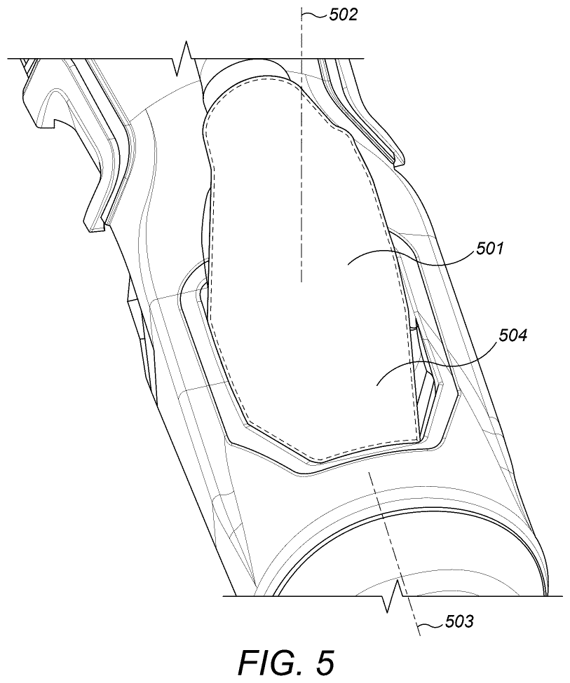

FIG. 5 illustrates the interior of the instrument body when the ultrasonic instrument is assembled as a whole. The transducer 501 is mounted transverse to the waveguide 404. The longitudinal axis of the transducer 502 is perpendicular to the longitudinal axis of the waveguide 503. Piezoelectric elements 504 of the transducer 501 may be formed of ceramic or another suitable material. For a rotating waveguide, when powered, the transducer converts alternating current into high frequency small amplitude oscillatory rotations of the waveguide 404 about its longitudinal axis 503. The rotatory oscillation of the waveguide is proportional to the voltage applied. For a linear waveguide, when powered, the transducer converts alternating current into high frequency small amplitude oscillatory vibrations of the waveguide 404 parallel to its longitudinal axis 503.

The waveguide and end effector element 405 are both stiff and rigidly attached to each other. There is no articulation between the waveguide and the end effector element. The end effector element 405 may be integrally formed with the waveguide. The end effector element 405 may be the tip of the waveguide 404. The waveguide is rigidly attached to the transducer 501. Utilising stiff components, rigidly attached to each other enables effective transfer of oscillation of the transducer 501 to the end effector element 405.

The second portion 401 comprises an instrument interface for engaging with and being driven by a corresponding robot arm interface of a surgical robot arm. The second portion comprises a drive mechanism (not visible on FIG. 4) for transferring drive from the instrument interface 406 to a shaft 407. The drive mechanism is housed in a casing 408. The second portion comprises the shaft 407 which connects to the casing 408 at its proximal end, and to an articulated end effector element 409 at its distal end.

Any suitable mechanism may be used to transfer drive to the end effector. As described above, the end effector element 405 preferably is rigidly attached to the waveguide 404 in order to most effectively transfer the ultrasonic energy from the transducer to the end effector element 405. Thus, the end effector as a whole does not have several degrees of freedom, for example the ability to rotate about pitch and yaw joints. The end effector element 409 moves with a single degree of freedom only. Specifically, the end effector element 409 may rotate with respect to the end effector element 405. The end effector element 409 may form an opposing jaw to the jaw 405. Thus, when the first and second portions of the ultrasonic instrument are assembled together, the upper jaw 409 may hinge relative to the lower jaw 405 so as to enable it to rotate in one rotational direction in order to open the jaws apart and to rotate in the opposing rotational direction to close the jaws together. In this way, the end effector can grasp and release tissue between the jaws.

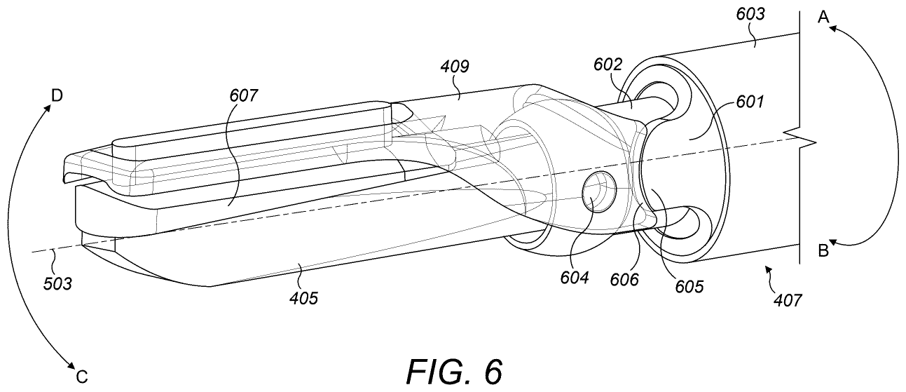

FIG. 6 illustrates an example distal end of the assembled ultrasonic instrument in more detail. The shaft 407 comprises an outer shaft 601, an inner shaft 602 and an insulating sleeve 603. The inner shaft, outer shaft and insulating sleeve are concentrically arranged. The outer shaft encompasses the inner shaft. The insulating sleeve encompasses the outer shaft. The inner shaft 602 is pivotally fixed to the end effector element 409. The outer shaft 601 is rotatable relative to the inner shaft 602. The drive mechanism transfers drive from the instrument interface 406 to rotation of the outer shaft 601. A cam mechanism at the distal end of the shaft 407 transfers this rotation of the outer shaft 601 to rotation of the end effector element 409 about the pivot 604. The outer shaft 601 and end effector element 409 abut each other at respective contacting surfaces 605, 606. They are thus in contact, but are not fixedly attached to each other. The contacting surfaces 605, 606 are cooperatively shaped. The contacting surfaces shown in FIG. 6 are curved, although a different shape may be used. Neither the end effector element 409 nor the outer shaft 601 has any freedom to move along the axial direction 503 of the shaft. This, combined with the curved shape of the contacting surfaces 605, 606 means that as the outer shaft 601 rotates in one rotational direction A about its longitudinal axis 503, one end of the contacting surface 605 of the outer shaft pushes against the end of the contacting surface 606 of the end effector element 409 that it abuts, causing the end effector element 409 to rotate about the pivot 604 in a rotational direction C so as to close the end effector element 409 towards the end effector element 405. Conversely, as the outer shaft 601 rotates about its longitudinal axis 503 in the opposing rotational direction B, the other end of the contacting surface 605 of the outer shaft pushes against the other end of the contacting surface 606 of the end effector element 409, causing the end effector element 409 to rotate about the pivot 604 in a rotational direction D so as to open the end effector element 409 away from the end effector element 405.

The end effector element 409 has a protective pad 607 attached along the surface of it which contacts the end effector element 405. This protective pad 607 protects the end effector element 409 from damage which may otherwise be caused by the oscillating waveguide 404 when the end effector elements 405 and 409 are closed together without any tissue being grasped between them. Suitably, this protective pad is fabricated from PTFE.

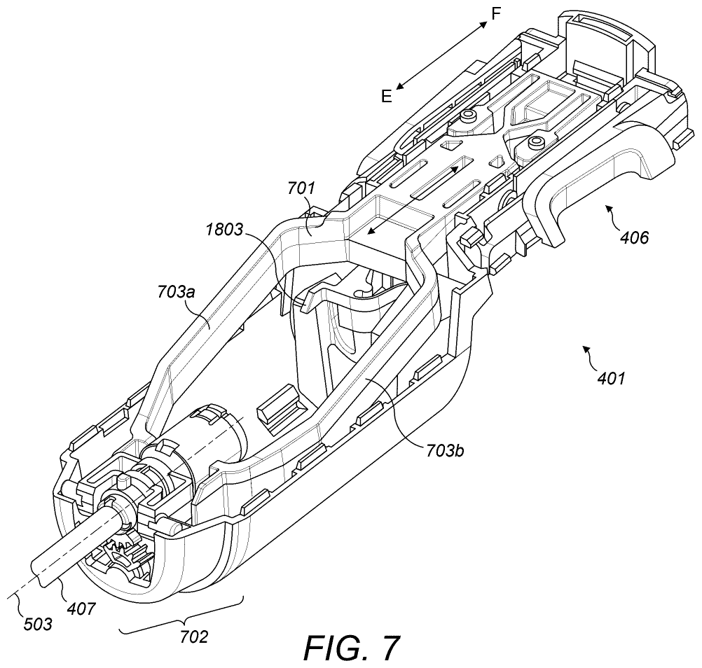

FIG. 7 illustrates the proximal end of the second portion of the ultrasonic instrument in more detail. The top half of the casing 408 has been omitted to reveal the interior of the second portion. The drive mechanism for transferring drive from the instrument interface 406 to the shaft 407 comprises a transmission structure 701 and a drive assembly 702. The transmission structure 701 spans the length of the proximal end of the second portion of the ultrasonic instrument up to the shaft 407. The transmission structure is a rigid component which transfers drive from the instrument interface elements to the drive assembly 702 by moving linearly in an axial direction. This axial direction is shown by the arrows E,F in FIG. 7. The axial direction is parallel to the longitudinal axis of the shaft 503. The transmission structure transfers the linear drive around other componentry internal to the proximal end of the assembled ultrasonic instrument. The transmission structure is shaped to accommodate the transducer 501 in the assembled ultrasonic instrument. This is by means of two arms 703a, 703b of the transmission structure which extend around a hollow shaped to encompass the transducer 501.

The drive assembly 702 is a compact structure in the axial direction of the instrument. The drive assembly 702 connects the transmission structure 701 to the shaft 407. The drive assembly 702 converts linear motion of the transmission structure 701 to rotational motion for driving the rotatable outer shaft 601.

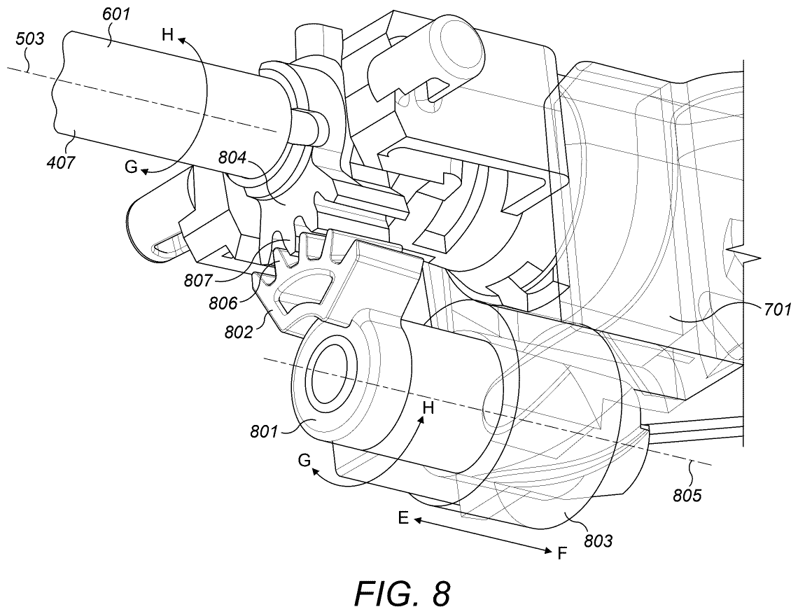

FIG. 8 illustrates the drive assembly 702 and the shaft 407 in more detail. The drive assembly 702 comprises a helical drive 801. The helical drive has a cylindrical shape. The longitudinal axis of the helical drive 805 lies parallel to the longitudinal axis of the shaft 503 in the assembled instrument. The longitudinal axis of the helical drive 805 is offset from the longitudinal axis of the shaft 503. The helical drive is slotted into a cylindrical drive 803 of the transmission structure. The helical drive 801 and cylindrical drive 803 are concentrically arranged. The helical drive 801 and cylindrical drive 803 share the same longitudinal axis 805. The exterior surface of the helical drive 801 is threaded. The interior surface of the cylindrical drive 803 of the transmission structure is threaded with a complementary thread. Thus, the helical drive 801 is in threaded engagement with the cylindrical drive 803 of the transmission structure. Thus, linear movement of the cylindrical drive 803 along the axial direction of the instrument causes the helical drive 801 to rotate about the longitudinal axis of the helical drive 805.

A drive gear 802 is rigidly attached to the helical drive. The drive gear 802 is a part gear which has teeth which extend partially around the exterior surface of the helical drive 801 proximal to the shaft 407. The drive gear 802 rotates about the longitudinal axis 805 of the helical drive 801 as the helical drive 801 rotates. The drive gear 802 meshes with a shaft gear 804. Shaft gear 804 is rigidly attached to the rotatable outer shaft member 601. The shaft gear 804 is a part gear which has teeth which extend partially around the exterior surface of the outer shaft 601. The shaft gear 804 rotates about the longitudinal axis 503 of the shaft as the drive gear 802 rotates. Movement of the drive gear 802 in one rotational direction causes the shaft gear 804 to rotate in the opposing rotational direction. Thus, linear motion of the transmission structure in a first linear direction F drives the drive gear 802 to rotate in a first rotational direction G, which drives the shaft gear 804 to rotate in a second rotational direction H which opposes the first rotational direction G, which drives the outer shaft 601 to rotate in the second rotational direction H. Whereas, linear motion of the transmission structure in a second linear direction E drives the drive gear 802 to rotate in the second rotational direction H, which drives the shaft gear 804 to rotate in the first rotational direction G, which drives the outer shaft 601 to rotate in the first rotational direction G.

Although FIG. 8 depicts the cylindrical drive 803 as surrounding the helical drive 801, the helical drive 801 may instead surround the cylindrical drive 803. In this case, the exterior of the cylindrical drive would be in threaded engagement with the interior of the helical drive.

The number of wraps of the thread around the circumference of the helical drive 801 and cylindrical drive 803 depends on the desired rotation of the end effector for a unit linear motion of the instrument interface elements. The tighter the thread, i.e. the greater the number of wraps of the thread around the circumference of the helical drive/cylindrical drive, the greater the rotational movement of the shaft and hence end effector elements per unit linear motion of the instrument interface elements. In the example of FIG. 8, the whole range of motion of the end effector element 409 is accommodated by a part rotation of the outer shaft 601. Thus, the drive gear and shaft gear are both part gears which extend sufficiently around the helical drive and outer shaft respectively to enable the transfer of motion from the helical drive to the outer shaft across the whole range of motion of the end effector element 409. Use of part gears enables a more compact arrangement in the plane perpendicular to the longitudinal axes 503 and 805 of the shaft and helical drive respectively. The teeth of the drive gear may extend less than 90° around the helical drive. The teeth of the shaft gear may extend less than 90° around the shaft.

Drive is transferred from the helical drive 801 to the outer shaft 601 in a plane perpendicular to the longitudinal axes of the helical drive 805 and the shaft 503. This enables the drive assembly to be very compact in the axial direction of the shaft. The following features about the arrangement of the instrument enable this compact arrangement. The longitudinal axes of the helical drive 805 and shaft 503 are offset. The helical drive 801, outer shaft 601, drive gear 802 and shaft gear 804 are all located in the same plane perpendicular to the longitudinal axes of the helical drive 805 and outer shaft 503. The teeth of the drive gear 806 extend in a direction perpendicular to the longitudinal axis of the helical drive 805. The teeth of the shaft gear 807 extend in a direction perpendicular to the longitudinal axis of the outer shaft 503. The teeth of the drive gear mesh with the teeth of the shaft gear in a direction perpendicular to the longitudinal axis of the helical drive.

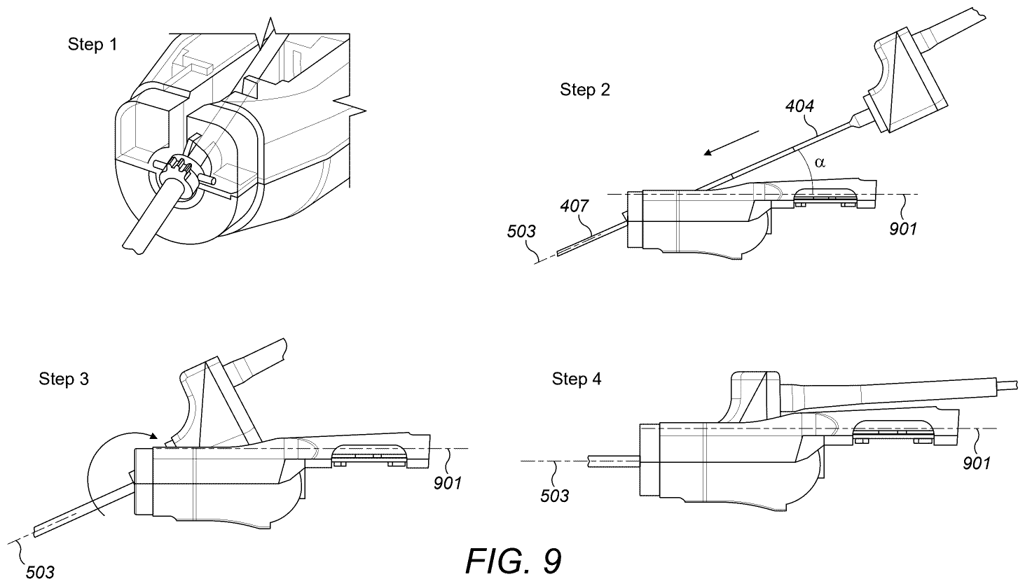

FIG. 9 illustrates a method of assembling the first and second portions of the ultrasonic surgical instrument described herein.

The shaft 407 is pivotable with respect to the casing 408 in order to allow the waveguide 404 of the first portion to be inserted into the shaft 407. Step 1 of FIG. 9 shows the waveguide 404 starting to be inserted into the shaft 407. The direction of insertion of the waveguide 404 into the shaft 407 is angled to the longitudinal axis of the instrument interface 901. As shown in step 2, this angle is α. α may be in the range 0°<α<450. α may be in the range 15°<α<250. Once the waveguide is fully inserted into the shaft, the first portion is rotated in the direction shown in step 3 to bring the longitudinal axis of the shaft 503 parallel with the longitudinal axis of the instrument interface 901. The first portion latches into place with the second portion. The final assembled position is shown in step 4.

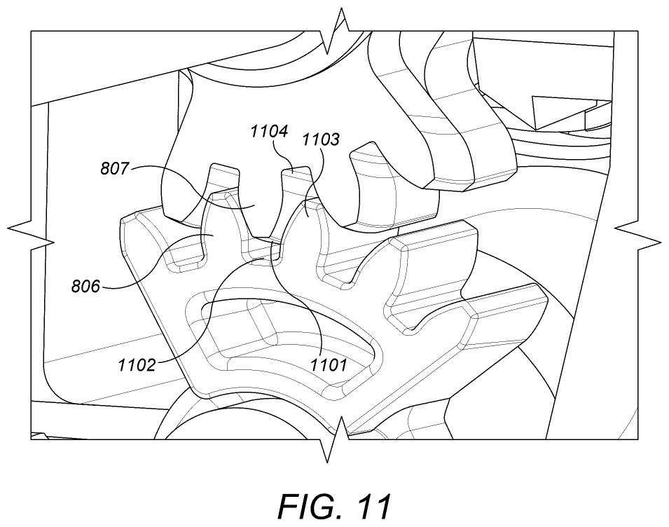

The shaft gear 804 is pivotable with respect to the drive gear 802 when the shaft gear and drive gear are meshed together. As can be seen in FIG. 11, each tooth 807 of the shaft gear extends only partway down the valley between adjacent drive gear teeth 806. The shaft gear teeth may have rounded ends 1101. This enables the ends 1101 of the shaft gear teeth to be angled relative to the valley floor 1102 between adjacent drive gear teeth whilst the shaft gear and drive gear are meshed together.

Additionally, or alternatively, each tooth 806 of the drive gear may extend only partway down the valley between adjacent shaft gear teeth 807. The drive gear teeth may have rounded ends 1103. This enables the ends 1103 of the drive gear teeth to be angled relative to the valley floor 1104 between adjacent shaft gear teeth whilst the shaft gear and the drive gear are meshed together. This aids accommodation of the angle of insertion a of the first portion into the second portion of the ultrasonic instrument.

As mentioned above, the surface of the end effector element 409 which contacts the end effector element 405 is covered in a protective pad 607. The protective pad is secured to the end effector element 409. Insertion of the waveguide 404 into the shaft 407 when the end effector element 409 is in a closed position could cause the protective pad 607 to be damaged by the end effector element 405 as the end effector element 405 is pushed into position. To avoid this problem, an alignment feature may be used such that the waveguide can only be inserted or removed from the shaft when the end effector elements are in an open configuration.

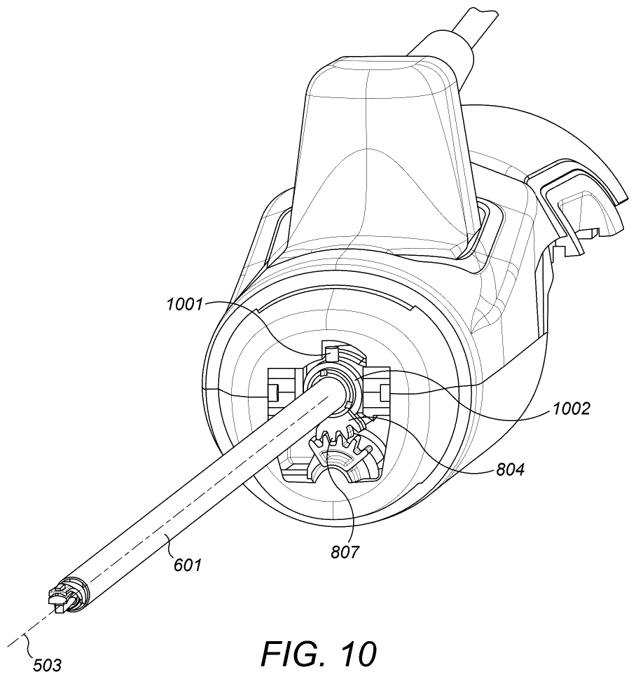

Referring to FIG. 10, the outer shaft 601 comprises a protrusion 1001 which extends in a direction perpendicular to the longitudinal axis of the shaft 503. In the example shown in FIG. 10, the protrusion 1001 is attached to the shaft gear 804. The shaft gear 804 comprises a ring which surrounds the outer shaft 601. The ring is in a plane perpendicular to the longitudinal axis of the shaft 503. The centre of the ring is a point on the longitudinal axis of the shaft 503. The teeth 807 of the shaft gear and the protrusion 1001 both extend from the ring in a direction perpendicular to the longitudinal axis of the shaft 503. The protrusion extends from the ring in a direction opposing the teeth of the shaft gear.

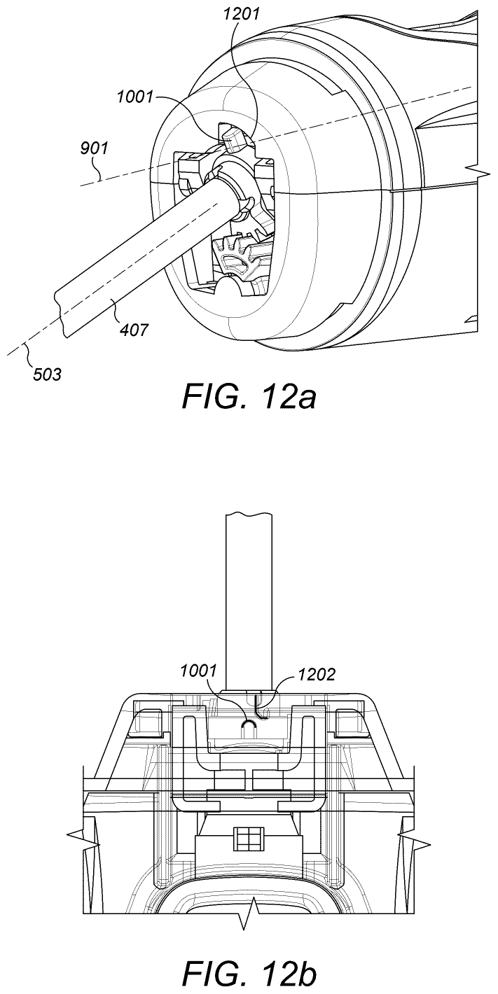

As illustrated in FIGS. 12a and 12b, the casing of the first portion comprises a notch 1201.

The notch 1201 constrains the protrusion 1001 when the shaft 407 is pivoted relative to the casing such that the longitudinal axis of the shaft 503 is angled relative to the longitudinal axis of the instrument interface 901, for example in the configuration shown in step 2 of FIG. 9 which is adopted whilst assembling the first and second portions together. When the shaft 407 is not pivoted relative to the casing, i.e. when the longitudinal axis of the shaft 503 is parallel to the longitudinal axis of the instrument interface 901, the protrusion 1001 is not constrained by the notch 1201. The shaft 407 is pivoted relative to the casing when the first portion is being inserted into or removed from the second portion.

The protrusion may be free to rotate within the notch 1201 about the longitudinal axis of the shaft 503 within a notch range. In this case, the rotatable range of the protrusion 1001 about the shaft axis 503 within the notch 1201 is limited by the length of the notch in the direction of rotation of the protrusion about the shaft axis 503. In other words, the circumferential length of the notch about the shaft axis 503. Since the protrusion 1001 rotates as the drive gear is driven, the position of the protrusion within the notch range corresponds to the opening angle of the end effector elements. When the protrusion is constrained by the notch, the end effector elements are in an open configuration. The whole of the notch range corresponds to opening angles between the end effector elements which are greater than 200.

Alternatively, the protrusion may not be free to rotate within the notch 1201 about the longitudinal axis of the shaft 503. Instead, the protrusion may have an interference fit with the notch 1201. In this case, when the protrusion is constrained within the notch, the end effectors have a specific opening angle between them. This specific opening angle is greater than 200.

In order to insert the first portion into the second portion, the shaft 407 is pivoted down away from the plane of the instrument interface. This is shown in step 2 of FIG. 9. The user may hold the first portion at the angle α to the longitudinal axis 901 of the instrument interface and insert the tip of the waveguide 404 into the shaft 407. The shaft 407 is only able to pivot if the protrusion 1001 is within the notch 1201. Thus, with the tip of the waveguide 404 in the shaft 407, the user rotates the first portion about the shaft axis 503 until the protrusion aligns with and passes into the notch 1201. FIG. 12b illustrates an alignment feature which may be used to aid this. There is a rib 1202 in the casing which the protrusion butts up against as the user rotates the first portion about the shaft axis 503. When the first portion is in a rotational position in which the protrusion is not aligned with the notch 1201, the protrusion 1001 butts up against the rib 1202 preventing the shaft from being pivoted down. When the first portion is in a rotational position in which the protrusion is aligned with the notch 1201, the protrusion is clear of the rib 1202, which enables the user to pivot the shaft down with the protrusion retained in the notch. The end of the rib 1202 which touches the protrusion is rounded. Similarly, the end of the protrusion 1001 which touches the rib 1202 is rounded. Thus, when the first portion is almost but not quite in a rotational position in which the protrusion is aligned with the notch, the contact of the rounded end of the protrusion 1001 with the rounded end of the rib 1202 nudges the protrusion into the correct rotational position to slot into the notch as the user tries to pivot the first portion down. Once the protrusion is retained in the notch, the shaft 407 is free to pivot relative to the casing. In the position in which the protrusion is retained in the notch, the end effector element 409 adopts an open configuration. The waveguide is then angled to enable it to be pushed into the shaft, causing the shaft 407 to pivot relative to the casing. The waveguide is pushed through the shaft until the transducer reaches the recess in the casing where it will be retained. This is shown in step 3 of FIG. 9. The tip of the waveguide exits through the distal end of the shaft to form the lower end effector element 405. Since the end effector element 409 is in an open configuration, the tip of the waveguide does not touch, and hence does not damage, the protective pad on the upper end effector element 409 as it is pushed into place.

In order to detach the first portion from the second portion, the shaft 407 is pivoted down away from the plane of the instrument interface as shown in step 3 of FIG. 9. As above, the shaft 407 is only able to pivot if the protrusion 1001 is within the notch 1201. Thus, the user rotates the first portion about the shaft axis 503 until the protrusion passes into the notch 1201 in the same manner as described above. Once the protrusion is retained in the notch, the shaft 407 is free to pivot relative to the casing. In the position in which the protrusion is retained in the notch, the end effector element 409 adopts an open configuration. The waveguide is then angled to enable the transducer to be removed from its seated position in the casing, causing the shaft 407 to pivot relative to the casing. The waveguide is then removed through the shaft. Since the end effector elements are opened away from each other, removal of the lower end effector element 405 does not touch, and hence does not damage, the protective pad on the upper end effector element 409.

Although in FIG. 10 the protrusion is shown as being attached to the shaft gear 804, it may instead be separately attached to the outer shaft 601.

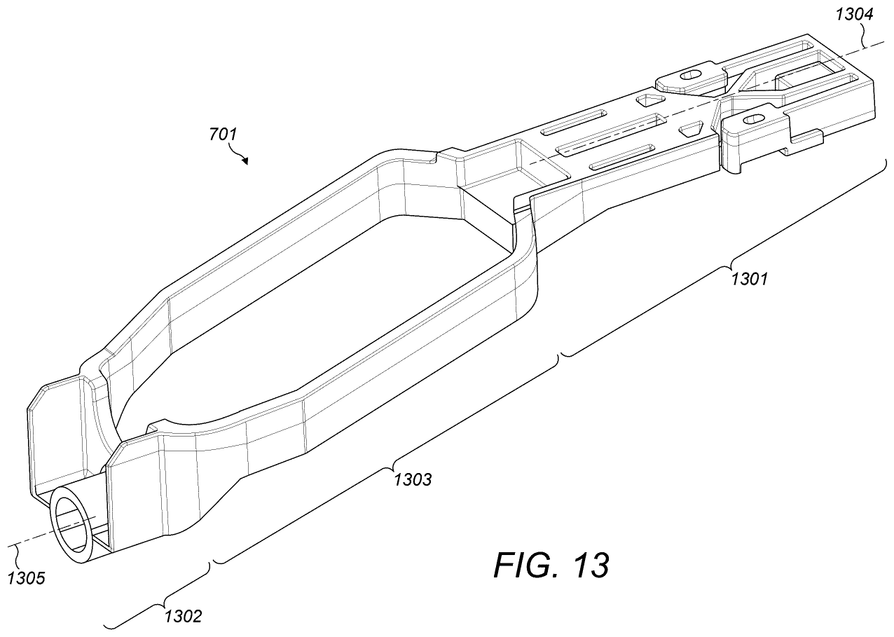

FIG. 13 illustrates the transmission structure 701 in isolation. It comprises three portions: a planar interfacing portion 1301, a drive portion 1302 proximal to the shaft, and a connector portion 1303 which connects the planar interfacing portion to the drive portion. The longitudinal axis 1304 of the planar interfacing portion is parallel to but offset from the longitudinal axis 1305 of the drive portion. The connector portion is itself linear, but angled relative to both the planar interfacing portion and the connector portion. Thus, the connector portion accommodates the change in plane of the transmission structure from that of the planar interfacing portion to that of the drive portion.

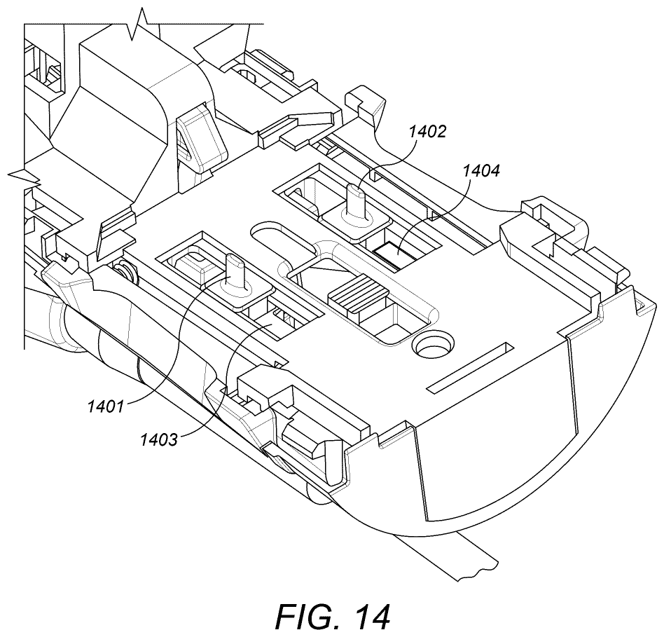

FIG. 14 illustrates the instrument interface of the instrument. Instrument interface elements 1401 and 1402 are linearly displaceable within slots 1403, 1404. The instrument interface elements depicted are plug-shaped and protrude from the surface of the instrument interface. When the instrument interface engages with a robot arm interface, the instrument interface elements engage with and are retained by cooperatively shaped drive assembly interface elements of the robot arm. For example, the drive assembly interface elements may be socket-shaped recesses, each of which retains a plug-shaped instrument interface element with an interference fit. Alternatively, the drive assembly interface elements may be plug-shaped and protrude from the surface of the robot arm interface, and the instrument interface elements socket-shaped recesses. In this case, the instrument interface elements retain the drive assembly interface elements when the instrument interface and robot arm interface are engaged.

As discussed above, to effectively transfer oscillation of the transducer 501 to the end effector element 405, the end effector element 405 is rigidly attached to the waveguide 404. The end effector element 405 is not articulated. There is only one degree of freedom of the end effector of the ultrasonic instrument shown in FIG. 6. This is opening and closing of the end effector, which is achieved by rotation of the top end effector element 409 alone with respect to the non-movable lower end effector element 405. The rotation of the end effector element 409 is driven by rotation of the outer shaft 601 about the longitudinal axis of the shaft 503. A single instrument interface element could be driven by the robot arm, and the drive of that single instrument interface element transferred to driving rotation of the outer shaft. However, when applying heat to tissue it can be useful to grip that tissue between the jaws of the end effector with a high grip force. The ultrasonic instrument described herein enables the end effector elements to grip tissue with a high grip force by coupling the drive of two instrument interface elements, as will now be described.

The instrument interface of FIG. 14 has two instrument interface elements 1401, 1402. Suitably, the instrument interface has only two instrument interface elements. Both of these instrument interface elements are driven by the drive assembly of the robot arm. The instrument interface elements may be independently driven by the drive assembly of the robot arm. Thus, a first drive assembly interface element may drive one of the instrument interface elements, and a second drive assembly interface element may drive the other instrument interface element. The first drive assembly interface element may be coupled to a first motor or actuator, and the second drive assembly interface element may be coupled to a second motor or actuator. The first motor/actuator is different to the second motor/actuator.

The transmission structure mechanically constrains the two instrument interface elements to move together. In doing so, the transmission structure itself is driven to move with the combined driving forces applied to the instrument interface elements by the robot arm. The driving force applied to the transmission structure is ultimately transmitted (with frictional losses) through to the gripping force of the end effector, via the drive assembly and outer shaft.

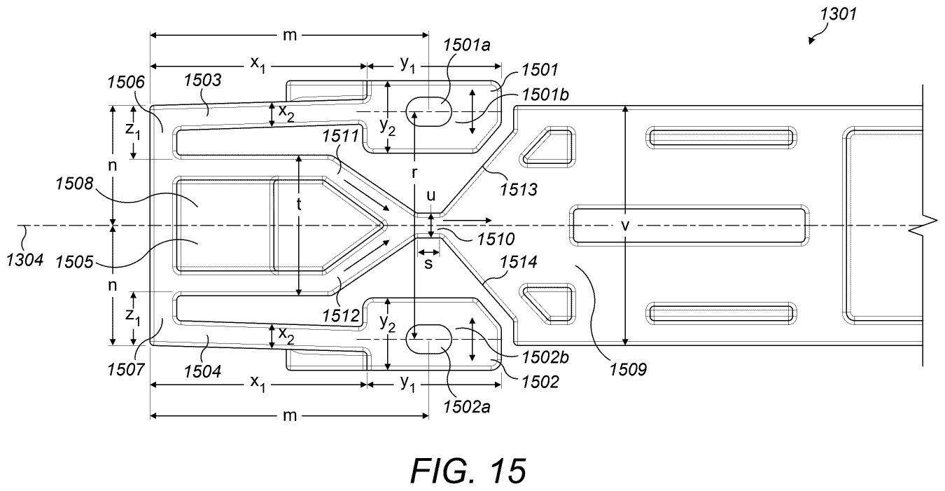

FIG. 15 illustrates the planar interfacing portion 1301 of the transmission structure. The transmission structure moves linearly in the directions E, F parallel to the longitudinal axis 1304 of the transmission structure. As the transmission structure moves linearly away from the shaft 407 in the direction F, it drives the helical drive to rotate in the rotational direction G (see FIG. 8) which drives the outer shaft 601 to rotate in the rotational direction H, which causes the end effector element 409 to rotate in the direction C (see FIG. 6) thereby causing the end effector elements to close together. Conversely, as the transmission structure moves linearly towards the shaft 407 in the direction E, it drives the helical drive to rotate in the rotational direction H which drives the outer shaft 601 to rotate in the rotational direction G, which causes the end effector element 409 to rotate in the direction D thereby causing the end effector elements to open apart.

Movement of the transmission structure in the direction F away from the shaft is the stronger direction of motion. Movement of the transmission structure in the direction E towards the shaft is the weaker direction of motion. This may be because the arms 703a, 703b of the connector portion 1303 (see FIG. 7) and the arms 1503, 1504 of the transmission structure (see FIG. 15) can withstand greater tension than buckling loads. The stronger direction of motion is chosen to be the direction which causes the end effector elements to close, and the weaker direction of motion is chosen to be the direction which causes the end effector elements to open. This is because it is more important that the gripping force of the end effector elements is high and accurately conveyed than the opening force of the end effector elements, since cutting and sealing operations are performed whilst the end effector elements are gripping tissue.

The transmission structure comprises two retaining structures 1501, 1502, each of which retains an instrument interface element (not shown). Instrument interface element 1401 is retained by retaining structure 1501. Instrument interface element 1402 is retained by retaining structure 1502. Each retaining structure is shaped to receive and retain the instrument interface element. Each retaining structure comprises a slot 1501a, 1502a surrounded by a frame 1501b, 1502b. The instrument interface element 1401, 1402 fits in the slot 1501a, 1502a with an interference fit. Thus, the drive force applied to the instrument interface element from the drive assembly of the robot arm is efficiently transferred to drive force applied to the transmission structure. Although each slot 1501a, 1502a shown in FIG. 15 is a hole, it may alternatively be socket-shaped, i.e. a recess with a base. The distance between the centres of the retaining structures 1501, 1502 perpendicular to the longitudinal axis 1304 of the transmission structure is r. The retaining structures 1501, 1502 are rigidly attached together. Each retaining structure is rigidly attached to the remainder of the transmission structure. The transmission structure as a whole is rigid with no articulatable portions. The transmission structure may be integrally formed. For example, the transmission structure may be moulded as a single part. Thus, the two instrument interface elements 1401, 1402 are mechanically constrained by the transmission structure to move together linearly in the direction E,F of the linear motion of the transmission structure.

Prior to attaching the ultrasonic instrument to the robot arm, the drive assembly interface elements of the robot arm which drive the instrument interface elements 1401, 1402 are aligned with each other in their range of travel. For example, the drive assembly interface elements may be driven to the mid-point of their range of travel. Although the instrument interface elements may be independently driven by the robot arm, they are driven in lock step. In other words, they are simultaneously driven in the same direction with the same force.

Each retaining structure 1501, 1502 is connected to the body 1505 of the transmission structure by an arm 1503, 1504. Each arm 1503, 1504 extends parallel to the longitudinal axis 1304 of the transmission structure. Each arm extends out from the retaining structure away from the shaft 407. The length of each arm x₁ in the direction parallel to the longitudinal axis of the transmission structure is greater than the length of each retaining structure y₁ in the same direction. For example, x₁>1.5y₁. Each arm is narrower x₂ in the direction perpendicular to the longitudinal axis of the transmission structure than the length of each retaining structure y₂ in the same direction. For example, x₂<2y₂. Each arm comprises a stem 1506, 1507 connecting the arm to the body 1505 of the transmission structure. The stem is perpendicular to the part of the arm which extends parallel to the longitudinal axis of the transmission structure. The stem has a length z₁ in the direction perpendicular to the longitudinal axis of the transmission structure. z₁>x₂. x₁>z₁. For example, x₁>2z₁. For example, x₁>4z₁. z₁>x₂ provides a gap in the direction perpendicular to the longitudinal axis 1304 of the transmission structure between each arm and retaining structure pair and the body of the transmission structure. This gap provides space for the arm to flex perpendicularly to the longitudinal axis of the transmission structure. Each arm has lower bending stiffness perpendicular to the longitudinal axis of the transmission structure than parallel to the longitudinal axis of the transmission structure. Thus, each arm flexes perpendicular to the direction of travel of the transmission structure, the direction of travel of the transmission structure being parallel to the longitudinal axis of the transmission structure. Since the transmission structure travels in the same direction as the instrument interface elements 1401, 1402, each arm flexes perpendicular to the direction of travel of the instrument interface elements.

The drive assembly interface elements which drive the instrument interface elements 1401, 1402 may be slightly misaligned with each other once they have been driven to the mid-point of their range of travel. The lateral flex of the arms 1503, 1504 enables the transmission structure to accommodate a degree of misalignment between the positions of the drive assembly interface elements. In other words, the arms flex so as to enable the instrument interface elements retained by the retaining structures to mate with the corresponding drive assembly interface elements of the robot arm. Thus, the instrument is able to be operatively engaged with the robot arm.

Manufacturing variation in any of the transmission structure, the instrument interface, or the robot arm interface may lead to the retaining structure, instrument interface element and drive assembly interface element not exactly aligning. The lateral flex of the arms 1503, 1504 enables the transmission structure to accommodate a degree of misalignment of the retaining structure, instrument interface element and drive assembly interface element perpendicular to the longitudinal axis 1304 of the transmission structure, and still enable the instrument interface and robot arm interface to operatively engage.

The drive assembly interface elements are driven in sync such that the same driving force is applied to each retaining structure via the instrument interface elements. Any variation or misalignment in the driving forces—i.e. asymmetry in the driving forces—applied by the drive assembly interface elements to the retaining structures via the instrument interface elements is accommodated by the arms 1503, 1504 flexing laterally. However, this introduces unwanted moments in the motion of the transmission structure which are not in the direction of linear motion of the transmission structure. The body 1505 of the transmission structure may be shaped so as to minimise the transmission of these unwanted moments through to the connector portion and drive portion of the transmission structure.

The body of the transmission structure comprises a first body part 1508 and a second body part 1509. The first body part and the second body part are non-overlapping in the direction of the longitudinal axis 1304 of the transmission structure. The second body part 1509 is closer to the shaft than the first body part 1508. The second body part 1509 lies wholly between the first body part 1508 and the shaft. The second body part is larger than the first body part. The second body part is longer than the first body part both in the direction of the longitudinal axis 1304 of the transmission structure, and perpendicular to it.

The first body part 1508 extends along the direction of the longitudinal axis 1304 of the transmission structure. The first body part 1508 is connected at the rear of the transmission structure to the arms 1503, 1504 via their stems. The first body part 1508 sits internal to the outer profile of the transmission structure. The arms 1503, 1504 are positioned at the exterior of the transmission structure, flanking the first body part. The first body part is connected at its other end to the second body part 1509 via a bridge 1510. The width of the first body part 1508 perpendicular to the longitudinal axis 1304 of the transmission structure tapers down to connect to one end of the bridge 1510. The width of the first body part 1508 tapers down to meet the bridge 1510 symmetrically about the longitudinal axis 1304 of the transmission structure.

The bridge 1510 lies on the longitudinal axis 1304 of the transmission structure and extends along the longitudinal axis of the transmission structure. The bridge 1510 is aligned with the retaining structures 1501, 1502 along the direction of the longitudinal axis 1304 of the transmission structure. The bridge 1510 is in the same plane as the retaining structures 1501, 1502 perpendicular to the longitudinal axis 1304 of the transmission structure. The bridge 1510 is connected to the first body part 1508 at one end and the second body part 1509 at the other end. The bridge 1510 is not directly connected to any other part of the transmission structure. The bridge 1510 does not contact the retaining structures 1501, 1502.

The second body part 1509 extends along the direction of the longitudinal axis 1304 of the transmission structure. The second body part 1509 is connected to the bridge 1510. The width of the second body part perpendicular to the longitudinal axis 1304 of the transmission structure tapers down to connect to the end of the bridge 1510. The width of the second body part 1509 tapers down to meet the bridge 1510 symmetrically about the longitudinal axis 1304 of the transmission structure. At its other end, the second body part 1509 is connected to the arms 703a, 703b of the connector portion of the transmission structure (see FIG. 7). The second body part 1509 is not directly connected to the retaining structures 1501, 1502. The second body part 1509 does not contact the retaining structures 1501, 1502.

The retaining structures 1501, 1502 are only directly connected to the arms 1503, 1504. The retaining structures 1501, 1502 do not contact any other part of the transmission structure. The arms 1503, 1504 are only directly connected to the retaining structures and the first body part 1508.

The length s of the bridge 1510 in the direction of the longitudinal axis 1304 of the transmission structure is shorter than the length y₁ of the retaining structures along the same direction. s<y₁. The width of the bridge 1510 u perpendicular to the longitudinal axis 1304 of the transmission structure is less than the width t of the first body part 1508 in the same direction. u<t. The bridge may be at least 5 times narrower than the first body part perpendicular to the longitudinal axis 1304. 5u<t. The width of the bridge 1510 u perpendicular to the longitudinal axis 1304 of the transmission structure is less than the width v of the second body part 1509 in the same direction. u<v. The bridge may be at least 10 times narrower than the second body part perpendicular to the longitudinal axis 1304. 10u<v. The width of the bridge 1510 u perpendicular to the longitudinal axis 1304 of the transmission structure is less than the width r between the retaining structures 1501, 1502. u<r. The bridge may be at least 8 times narrower than the distance r between the retaining structures. 8u<r. The bridge may be at least 10 times narrower than the distance r between the retaining structures. 10u<r.

The distance m between the rear edge of the transmission structure and the centre of each retaining structure parallel to the longitudinal axis 1304 of the transmission structure is greater than the width of the bridge u perpendicular to the longitudinal axis of the transmission structure. Suitably, m>3u. Suitably, m<9u. The thickness of the transmission structure perpendicular to the plane shown in FIG. 15 is p. The distance m is greater than the thickness p. Suitably, m>3p. Suitably, m<9p. The distance n between the longitudinal axis 1304 of the transmission structure and the exterior edge of the arm at the outer edge of the transmission structure perpendicular to the longitudinal axis of the transmission structure is greater than the length of each arm x₂ in the direction parallel to the longitudinal axis of the transmission structure. Suitably, n>10x₂. Suitably, n<20x₂. The distance n is greater than the thickness p of the transmission structure perpendicular to the plane of the transmission structure shown in FIG. 15. Suitably, n>10p. Suitably, n<20p.

The tapered end of the first body portion 1508 has diagonal sides 1511 and 1512. These diagonal sides extend from sides of the first body portion 1508 which are parallel to the longitudinal axis 1304 of the transmission structure and separated by the width t, down to meet the bridge 1510. This arrangement minimises unwanted lateral moments from being transferred from the first body portion 1508 through to the second body portion 1509. Similarly, the tapered end of the second body portion 1509 has diagonal sides 1513 and 1514. These diagonal sides extend from sides of the second body portion 1509 which are parallel to the longitudinal axis 1304 of the transmission structure and separated by the width v, down to meet the bridge 1510. This arrangement minimises unwanted lateral moments from being transferred from the second body portion 1509 through to the first body portion 1508.

The transmission structure is symmetrical about its longitudinal axis 1304. The transmission structure is reflectively symmetrical about its longitudinal axis 1304.

It is useful to design robotic surgical instruments to be backdriveable. In other words, the end effector elements can be manipulated (such as opening or closing jaws) in order to move the end effector elements at the instrument interface. This aids the bedside team in preparing an instrument for assembly to the robot arm if, for example, the instrument has to be in a particular configuration (typically straight with the end effector elements closed) in order to operatively engage it with the robot arm. It is also a useful safety mechanism for the bedside team to be able to manually open the end effector elements in case of a major error with the surgical system.

The manner in which articulation of the end effector of the ultrasonic instrument described herein is driven causes it to not be backdriveable. There are two primary reasons for this. Firstly, there is a much higher gear ratio in the described instrument than typical cable driven instruments. In a typical cable driven instrument, moving the instrument interface elements across their full range of motion would cause the end effector elements of the instrument to open to approximately 180°. By comparison, moving the instrument interface elements across the same full range of motion with the described ultrasonic instrument drives a maximum opening angle of the end effector elements of approximately 60°. Thus, approximately three times as much force would need to be applied to actuate the end effector elements of the ultrasonic instrument in order to cause a corresponding motion of the instrument interface element as would be required for a typical cable driven instrument. Secondly, the helical drive described herein is not as efficient at transferring drive force as a typical cable driven instrument. It has higher losses, for example due to static friction. This means additional force is required to be applied to actuate the end effector elements in order to overcome the losses of the helical drive. The combination of these factors means that so much force would need to be applied to the end effector elements to backdrive the instrument, that that force would likely damage the end effector elements. For example, it might cause them to snap.

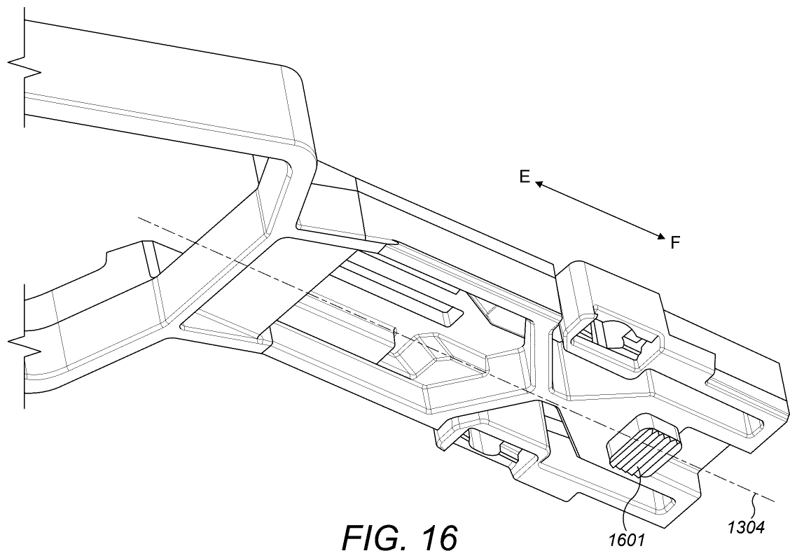

FIG. 16 illustrates the underneath of the transmission structure shown in FIG. 13. A slider projection 1601 can be seen on the transmission structure. The slider projection 1601 may protrude from the plane of the exterior surface of the transmission structure. The purpose of the slider projection 1601 is to enable a user to manually push the transmission structure linearly so as to allow manual articulation of the end effector. In particular, a user can push the slider projection 1601 in the direction E so as to drive opening of the end effector. This would cause the end effector to drop whatever it was gripping (for example tissue), allowing the instrument to be removed from the surgical site without causing further damage. This is useful in an emergency situation where the surgical instrument needs to be extracted from the surgical site, but a failure with the surgical robotic system means this extraction cannot be driven via the robot arm as normal and instead needs to be manually achieved.

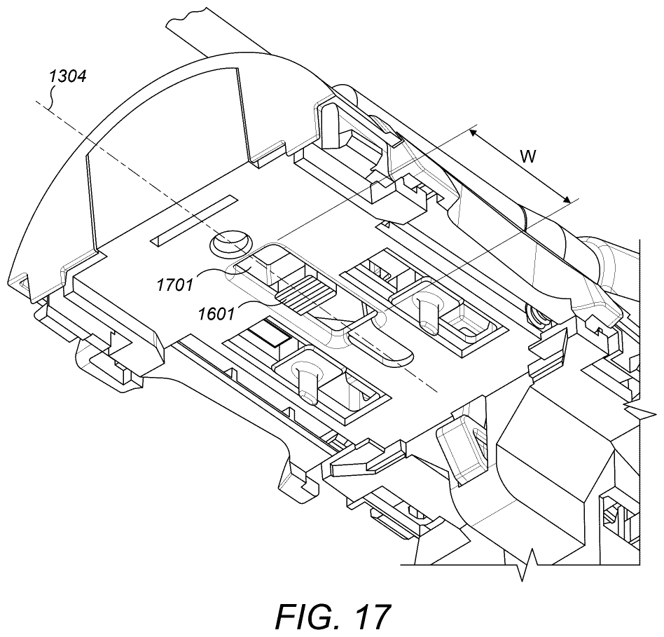

FIG. 17 shows the instrument interface of the assembled surgical robotic instrument. The slider projection 1601 is exposed on the exterior of the casing of the instrument interface. The slider projection is accessible by a user when the surgical robotic instrument is assembled. The casing of the instrument interface has a hollow 1701 through which the slider projection protrudes and/or the user is able to access the slider projection. The hollow extends along the direction of linear motion of the transmission structure a length w which is the same as or exceeds the range of motion of the transmission structure. Perpendicular to the direction of linear motion of the transmission structure, the hollow extends at least the length of the slider projection in that same direction. Thus, the user is able to manually push the transmission structure linearly across its entire range of motion by pushing the slider projection through the hollow 1701.

In the example shown in FIG. 16, the slider projection 1601 is located along the longitudinal axis 1304 of the transmission structure. It is located on the planar interfacing portion 1301 of the transmission structure. It is located between the retaining structures 1501 and 1502 which receive the instrument interface elements. Although shown in this location in FIGS. 16 and 17, it could alternatively be placed anywhere else on the transmission structure where it is accessible on the exterior of the instrument to a user when the instrument is assembled. Suitably, the slider projection comprises a textured surface. This is to aid movement of it by a user.

As described above, for the ultrasonic instrument to be operatively engaged on the robot arm, the first portion of the instrument needs to be received in and properly seated in the second portion. The assembled ultrasonic instrument must then be properly docked on the robot arm via the operative engagement of the instrument interface to the robot arm interface. The following describes latches which ensure that the ultrasonic instrument is operatively engaged on the robot arm. In other words, the latches ensure that both: (i) the ultrasonic instrument is correctly assembled, i.e. the first portion is properly retained in the second portion, and (ii) the ultrasonic instrument is correctly docked to the robot arm.

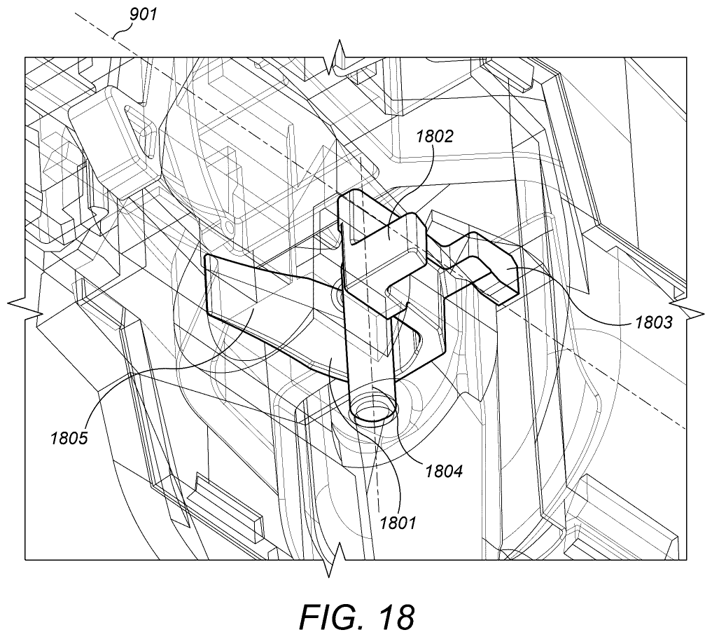

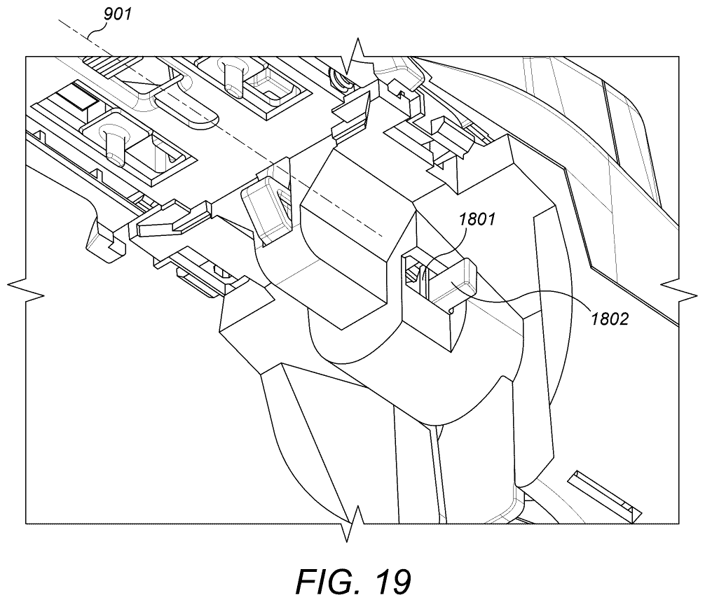

FIGS. 18 and 19 illustrate an example latch 1801 located on the second portion of the instrument which prevents the instrument from being able to be docked onto the robot arm unless the two portions of the instrument have been correctly assembled. In other words, the latch permits the instrument to be docked onto the robot arm only if the first portion of the instrument has been properly received by the second portion.

FIG. 18 illustrates the latch 1801 with the casing partially omitted thereby enabling the full shape and position of the latch to be seen. FIG. 19 illustrates the latch with the casing in place.

The latch is located on the second portion of the instrument. The latch is located such that a portion of it is exposable at the surface of the instrument interface which engages the robot arm interface. The latch is located such that it is actuatable by the first portion when the first portion is received by the second portion.

The latch is moveable between an open position and a closed position. FIGS. 18 and 19 illustrate the latch in the open position. In the open position, the latch prevents the instrument interface from operatively engaging with the robot arm interface. For example, in the open position the latch may comprise a prong 1802 which protrudes from the surface of the instrument interface which engages the robot arm interface, thereby physically blocking the instrument interface from engaging with the robot arm interface. The latch adopts the open position when the first portion and second portion of the instrument are separated from each other.

In the closed position, the latch 1801 permits the instrument interface to operatively engage with the robot arm interface. For example, in the closed position the latch 1801 may be wholly housed within the instrument, such that no part of the latch protrudes from the surface of the instrument interface which engages the robot arm interface. Thus, no part of the latch physically blocks the instrument interface from engaging with the robot arm interface. Thus, in the prong example above, the prong 1802 of the latch 1801 is fully housed within the instrument in the closed position. The latch 1801 adopts the closed position when the first portion is received in the casing of the second portion. This is achieved by the action of the first portion being received in the casing of the second portion actuating the latch causing it to move from the open position to the closed position. For example, the first portion may push on the latch causing it to move from the open position to the closed position.

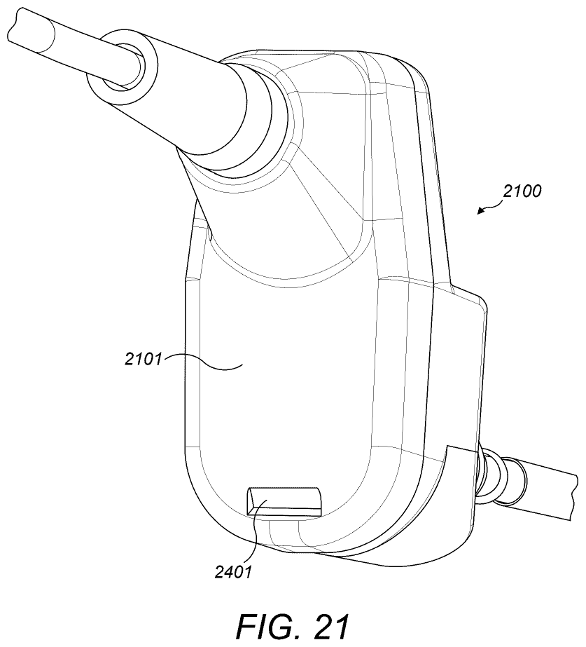

In the example shown in FIGS. 18 and 19, the latch 1801 is moveable. It rotates about a rotation axis 1804. In the example shown, this rotation axis is perpendicular to the longitudinal axis of the instrument interface 901. The latch comprises a second prong 1803. In the open position of the latch 1801, the second prong 1803 protrudes out from a surface of the casing of the second portion which abuts the first portion when the first portion is received in the casing. This can be seen in FIG. 7. Second prong 1803 protrudes into the cavity of the casing of the second portion which receives the first portion. The first prong 1802 and second prong 1803 are cooperatively arranged such that when the first portion is received in the second portion, the first portion pushes against the second prong, which causes the latch to rotate about axis 1804. This rotation causes the first prong 1802 to become fully housed in the instrument, thereby no longer obstructing the instrument interface and robot arm interfaces from operatively engaging. FIG. 21 illustrates the instrument body 2100 of the first portion. The exterior surface 2101 of the instrument body may push against the second prong 1803 as the instrument body of the first portion is received in the second portion, causing the latch 1801 to rotate about axis 1804.

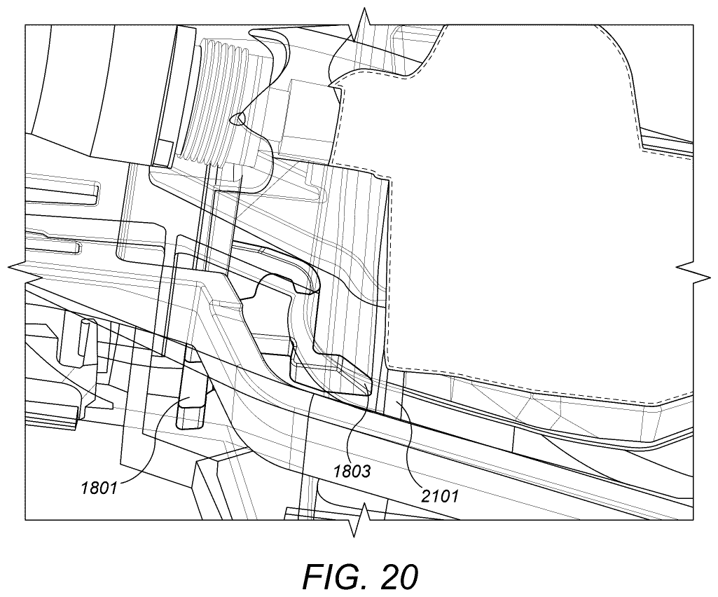

The first prong 1802 and second prong 1803 may be rigidly attached to each other. The whole of the latch 1801 may be a single rigid structure. The latch may be integrally formed. The first prong 1802 and second prong 1803 may protrude in different directions to each other. They thus protrude from different surfaces of the casing in the open position of the latch. In the figures shown, the first and second prong protrude perpendicularly to each other. They thus protrude out of perpendicular surfaces of the casing in the open position of the latch. In the open position of the latch, the first prong 1802 protrudes out of the surface of the instrument interface which engages the robot arm interface, and the second prong 1803 protrudes out of the surface of the second portion which abuts the first portion when it receives the first portion. Each of the first and second prong protrude in a direction perpendicular to the rotation axis of the latch 1804. The second prong 1803 protrudes in a direction parallel to the longitudinal axis of the instrument interface 901. The second prong 1803 terminates in a wedge which contacts the first portion when the first portion is received in the casing. The wedge shape is most easily seen in FIG. 20.

The latch may be biased into the open position. For example, latch 1801 may comprise a flexible portion 1805 which bends when the latch 1801 moves to the closed position. The flexible portion 1805 is in a bent configuration when the latch 1801 is in the closed position, and in a straight, non-bent configuration when the latch 1801 is in the open position. The flexible portion 1805 thus biases the latch 1801 to the open position. The latch may be biased into the open position by another feature. For example, the latch 1801 may comprise a spring which is compressed in the closed position, and uncompressed in the open position. Thus, the spring causes the latch 1801 to be biased into the open position.

FIGS. 18 and 20 show the two prongs protruding perpendicularly to each other. Alternatively, the two prongs may protrude at any angle to each other than is greater than 0°. In other words, the two prongs protrude at a non-parallel angle. The figures show the two prongs protruding perpendicularly to the latch rotation axis 1804. Alternatively, each of the two prongs may protrude at any angle to the latch rotation axis 1804 greater than 0°. In other words, the two prongs protrude at an angle non-parallel to the latch rotation axis 1804. The figures show the latch rotation axis 1804 to be perpendicular to the longitudinal axis of the instrument interface 901. Alternatively, the latch rotation axis 1804 could be at any other angle to the longitudinal axis of the instrument interface 901.

The latch 1801 shown in FIGS. 18 to 20 moves by rotation about a latch rotation axis 1804. Alternatively, the latch may move by linear translation. For example, the latch may be captive within a channel along which it can linearly move. When the first portion is received in the second portion, the first portion may push against the second prong causing the latch to move linearly. This translation may cause the first latch to become fully housed in the instrument, thereby not obstructing the instrument interface and robot arm interfaces from operatively engaging.

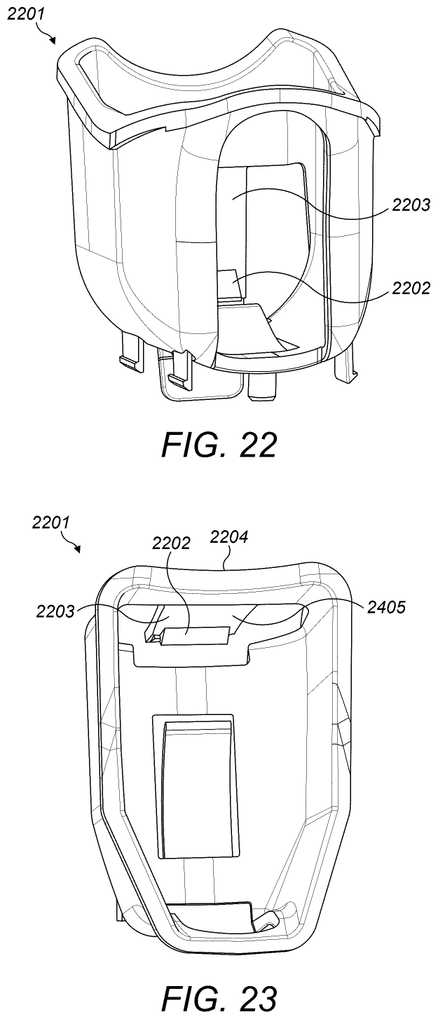

FIGS. 22, 23 and 24 illustrate a second latch cooperatively formed by the interaction between the meeting surfaces of the instrument body of the first portion and the casing of the second portion when the assembled instrument is operatively engaged with the robot arm. The second latch is a lockable latch which locks the first and second portion of the instrument together when the instrument is operatively engaged with the robot arm. In other words, the second latch prevents the first and second portions from detaching when the instrument interface is engaged with the robot arm interface.

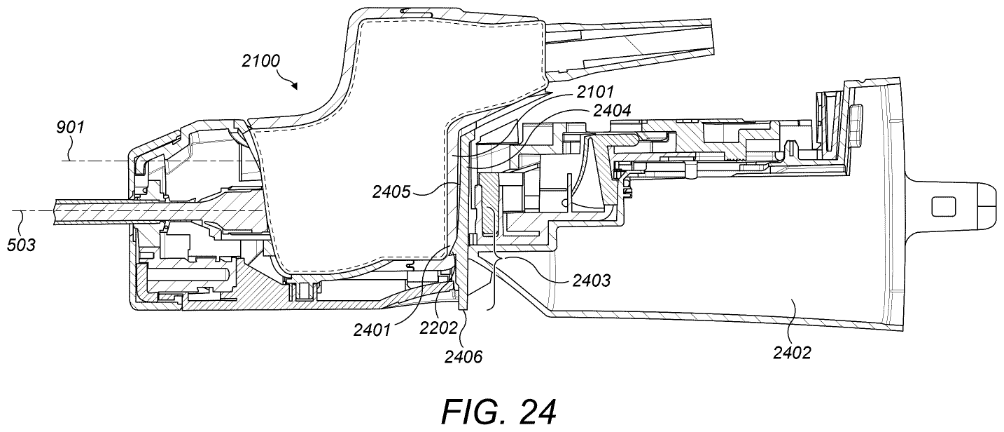

FIGS. 22 and 23 illustrate casing 2201 of the second portion which receives the instrument body 2100 of FIG. 21. FIG. 24 illustrates a cross section of the assembled instrument attached to the robot arm.

The casing 2201 of the second portion comprises a surface feature on its internal face which abuts the external face 2101 of the instrument body when the instrument body is received in the casing 2201. That surface feature is cooperatively shaped with a corresponding surface feature on the external face 2101 of the instrument body with which it engages when the first portion is received in the casing.

The surface features illustrated in FIGS. 21 to 24 are a notch 2401 on the external face of the instrument body, and a complementary shaped nib 2202 on the internal face of the casing 2201. The nib 2202 is receivable in the notch 2401 to form the lockable latch.

When the instrument is docked onto the robot arm with the instrument and robot arm interfaces operatively engaged, the robot arm butts up against the casing 2201 in such a way as to prevent the latch from detaching. In the example shown in FIG. 24, the robot arm 2402 contacts an exterior surface 2404 of the casing 2202 in the region 2403. The exterior surface 2404 of the casing opposes the interior surface 2405 of the casing which comprises the surface features which forms the lockable latch with the instrument body. In the example shown in FIG. 24, the casing is planar in the vicinity of the lockable latch. The instrument body is also planar in the vicinity of the lockable latch. Both the planar surface of the casing and the planar surface of the instrument body extend in the same direction. This direction is transverse to the longitudinal axis 503 of the shaft and transverse to the longitudinal axis 901 of the instrument interface 901. This direction may be perpendicular to the longitudinal axis 503 of the shaft and perpendicular to the longitudinal axis of the instrument interface 901. When the instrument is docked on the robot arm, the robot arm abuts the rear exterior surface 2404 of the casing which pushes the interior surface 2405 of the casing against the exterior surface 2101 of the instrument body. This action maintains the latch in the locked position. It prevents the latch from detaching and thus prevents the first and second portions of the instrument from detaching whilst the instrument is operatively engaged on the robot arm.

The casing 2201 may further comprise a lever 2203. The lever aids disassembly of the assembled instrument into the first and second portions when the instrument is not docked on the robot arm. The lever 2203 is on the face of the casing which interacts with the instrument body external surface 2101. The surface feature of the casing which forms the lockable latch, such as nib 2202, may be located on the lever 2203. The lever 2203 is bendable relative to the remainder of the casing 2201. The lever 2203 may be bendable away from the remainder of the casing 2201.

When the instrument is not attached to the robot arm, the user is able to bend the lever away from the remainder of the casing 2201. This action detaches the cooperating surface features of the instrument body and casing. In the example of FIGS. 22 to 24, it detaches the nib 2202 from the notch 2401. This detaches the instrument body from being locked in the casing. The user is then able to lift the first portion free of the second portion.

When the instrument is attached to the robot arm, the lever 2203 cannot be bent away from the remainder of the casing 2201. This is prevented by the robot arm abutting the lever 2203. When the instrument is docked on the robot arm, the lever 2203 is pushed by the robot arm such that the two cooperating surface features of the latch are pushed together in a locked configuration. For example, the nib is locked into the notch. The lever is not able to be actuated. This thereby prevents the instrument body from detaching from the casing.

The lockable second latch thus prevents unwanted decoupling of the surgical instrument from the robot arm during surgical use.

The lever 2203 protrudes out from the profile of the casing as shown at reference numeral 2406 of FIG. 24. This aids the user in gripping the lever to actuate it.

The example in FIGS. 21 to 24 has a nib and notch as the complementary surface features on the instrument body external face and casing internal face which cooperatively form the lockable latch. The nib has a convex wedge shape, and the notch a complementary concave wedge shape. Alternatively, any other complementary surface features may be used, for example a rounded pin may be used on the casing, and a complementary rounded recess on the instrument body.

The example in FIGS. 21 to 24 locates the lockable latch on a mating surface of the instrument body and casing which extends in a direction perpendicular to the longitudinal axis of the shaft 503. Alternatively, the lockable latch may be located on a different mating surface of the instrument body and casing. For example a surface which runs parallel to the longitudinal axis of the shaft 503.

The instrument described herein drives opening/closing of the end effector elements via rotation of outer shaft 601. However, rotation of the outer shaft 601 may alternatively drive any other degree of freedom of the end effector. For example, it could drive rotation of the end effector about the longitudinal axis of the shaft 503.

The instrument described herein drives the end effector to actuate via rotation of outer shaft 601. Alternatively, drive may be transferred to the end effector from the transmission structure via a different mechanism. For example, a cable extending down the interior of the shaft of the instrument may be used to transfer the linear motion of the transmission structure to the end effector to drive actuation of the end effector. As another example, the shaft may be driven axially along its longitudinal axis to drive actuation of the end effector. The instrument described herein transfers drive from the robot arm interface to the instrument interface via linear motion of drive assembly interface elements of the robot arm interface which drive corresponding linear motion of the instrument interface elements that they cooperatively engage. However, drive may alternatively be transferred from the robot arm interface to the instrument interface via a different mechanism. For example, rotational drive may be transferred from the robot arm interface to the instrument interface. This may be implemented, for example, by drive assembly interface elements which rotationally engage with corresponding instrument interface elements. For example, both the drive assembly interface elements and instrument interface elements may be rotatable spools. The rotational motion of the instrument interface element spools may be converted to linear drive of the transmission structure via cables and/or gears.

The first portion of the instrument described herein comprises an instrument body which houses a transducer for converting electrical energy to oscillation of the waveguide. The instrument is thus an ultrasonic instrument. However, the instrument body may alternatively house a different component and the rod down the interior of the shaft of the instrument have a different purpose. The remainder of the instrument may be as described above, however it would then be a different, non-ultrasonic, surgical instrument. For example, the instrument body may house a cable for feeding electrical energy to a conductive conduit (such as a rod or cable) which extends down the interior of the instrument shaft. In this case the instrument is an electrosurgical instrument.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

### ANNEX

1. A robotic surgical instrument comprising:
   a first portion comprising:
      an end effector;
      an instrument body; and
      a rod connected to the end effector at a distal end, and to the instrument body at a proximal end; and a second portion separable from the first portion, the second portion comprising:
   an instrument interface configured to engage with a corresponding robot arm interface of a surgical robot arm; and
   a casing configured to receive the first portion;
wherein, when the first portion is received in the casing, an external face of the instrument body abuts an internal face of the casing, the external face of the instrument body and the internal face of the casing cooperatively forming a lockable latch which prevents the first portion from detaching from the second portion when the instrument interface is engaged with the robot arm interface.
2. A robotic surgical instrument according to paragraph 1, wherein the external face of the instrument body comprises a notch, and the internal face of the casing comprises a nib, the nib receivable in the notch to form the lockable latch.
3. A robotic surgical instrument according to paragraph 2, wherein the internal face of the casing comprises a lever, the nib being located on the lever, the lever operable to, when pulled away from the instrument body, detach the nib from the notch thereby detaching the first portion from the casing.
4. A robotic surgical instrument according to paragraph 3, the lever being further operable to, when pushed towards the instrument body, lock the nib into the notch thereby preventing the first portion from detaching from the casing.
5. A robotic surgical instrument according to paragraphs 3 or 4, wherein the lever protrudes out from the profile of the casing.
6. A robotic surgical instrument according to any of paragraphs 1 to 5, wherein the external face of the instrument body and the internal face of the casing primarily extend in a plane transverse to the longitudinal axis of the rod.
7. A robotic surgical instrument according to any of paragraphs 1 to 6, wherein the external face of the instrument body and the internal face of the casing primarily extend in a plane perpendicular to the longitudinal axis of the rod.
8. A robotic surgical instrument comprising:
   a first portion comprising:
      an end effector;
      an instrument body; and
      a rod connected to the end effector at a distal end, and to the instrument body at a proximal end; and
a second portion separable from the first portion, the second portion comprising:
   an instrument interface configured to engage with a corresponding robot arm interface of a surgical robot arm;
   a casing configured to receive the first portion; and
   a moveable latch moveable between an open position and a closed position, wherein in the open position the moveable latch prevents the instrument interface from operatively engaging with the robot arm interface, and in the closed position the moveable latch permits the instrument interface to operatively engage with the robot arm interface, wherein the moveable latch adopts the open position when the second portion is separated from the first portion, and the moveable latch moves to the closed position when the first portion is received in the casing of the second portion.
9. A robotic surgical instrument according to paragraph 8, wherein in the closed position the moveable latch is fully housed within the robotic surgical instrument, and in the open position a first prong of the moveable latch protrudes out from the surface of the instrument interface which engages the robot arm interface.
10. A robotic surgical instrument according to paragraph 9, wherein in the open position a second prong of the moveable latch protrudes out from a surface of the casing which abuts the first portion when the first portion is received in the casing.
11. A robotic surgical instrument according to paragraph 10, wherein the first and second prongs are cooperatively arranged such that when the first portion is received in the casing, the first portion pushes against the second prong, which causes the first prong to become fully housed in the robotic surgical instrument, thereby permitting the instrument interface to operatively engage with the robot arm interface.
12. A robotic surgical instrument according to paragraph 10 or 11, wherein the first and second prongs of the moveable latch are rigidly attached to each other.
13. A robotic surgical instrument according to any of paragraphs 8 to 12, wherein the moveable latch is a rotatable latch.
14. A robotic surgical instrument according to paragraph 13, wherein the moveable latch rotates about a rotation axis perpendicular to the longitudinal axis of the rod.
15. A robotic surgical instrument according to any of paragraphs 10 to 14, wherein the first prong protrudes in a direction perpendicular to the second prong.
16. A robotic surgical instrument according to paragraph 14 or 15, wherein the first and second prongs each protrude in a direction perpendicular to the rotation axis.
17. A robotic surgical instrument according to any of paragraphs 10 to 16, wherein the second prong terminates in a wedge which contacts the first portion when the first portion is received in the casing.
18. A robotic surgical instrument according to any of paragraphs 8 to 17, wherein the moveable latch is biased to the open position.
19. A robotic surgical instrument according to any of paragraphs 1 to 18, wherein the robotic surgical instrument is an ultrasonic instrument, and the instrument body comprises a transducer.
20. A robotic surgical instrument according to any of paragraphs 1 to 19, wherein the end effector is an articulated end effector, the instrument interface is configured to be driven by a drive assembly of the robot arm interface, and the instrument interface comprises a drive mechanism configured to transfer drive from the drive assembly of the robot arm interface to drive articulation of the articulated end effector.
21. A robotic surgical instrument comprising:
   an articulated end effector;
   an instrument interface for engaging with and being driven by a corresponding robot arm interface of a surgical robot arm;
   a drive mechanism connected to the instrument interface, the drive mechanism comprising a transmission structure configured to transfer drive by moving linearly; and
   a shaft connected to the drive mechanism at a proximal end and the articulated end effector at a distal end, the shaft configured to transfer drive from the drive mechanism to the articulated end effector;
   wherein the instrument interface comprises two instrument interface elements, each instrument interface element driveable by a respective drive assembly interface element of the robot arm interface, the two instrument interface elements mechanically constrained by the transmission structure to move together such that when both are driven the combined driving forces applied to them drive the transmission structure to move linearly.
22. A robotic surgical instrument according to paragraph 21, wherein the two instrument interface elements are mechanically constrained by the transmission structure to move together linearly in the direction of the linear motion of the transmission structure.
23. A robotic surgical instrument according to paragraph 21 or 22, wherein the transmission structure comprises two retaining structures, each retaining structure shaped to retain one of the two instrument interface elements, the two retaining structures being rigidly attached together.
24. A robotic surgical instrument according to paragraph 23, wherein the two retaining structures are integrally formed.
25. A robotic surgical instrument according to any of paragraphs 21 to 24, wherein each retaining structure comprises a slot for receiving a plug-shaped instrument interface element.
26. A robotic surgical instrument according to any of paragraphs 21 to 25, wherein each retaining structure is connected to a body of the transmission structure by an arm, the arm configured to flex perpendicularly to the direction of the linear motion of the transmission structure.
27. A robotic surgical instrument according to paragraph 26, wherein each arm extends along the direction of the linear motion of the transmission structure, the arm being longer in the direction of the linear motion of the transmission structure than the retaining structure, the arm having a lower bending stiffness perpendicular to the direction of the linear motion of the transmission structure than parallel to the direction of the linear motion of the transmission structure.
28. A robotic surgical instrument according to paragraph 26 or 27, wherein the body of the transmission structure comprises a first body part and a second body part, only the first body part being connected to the two arms, the first and second body parts connected to each other by a bridge.
29. A robotic surgical instrument according to paragraph 28, wherein the first and second body parts are non-overlapping in the direction of the linear motion of the transmission structure, the second body part being closer to the shaft than the first body part.
30. A robotic surgical instrument according to paragraph 28 or 29, wherein the width of the first body part perpendicular to the direction of the linear motion of the transmission structure tapers down to connect to one end of the bridge, and the width of the second body part perpendicular to the direction of the linear motion of the transmission structure tapers down to connect to the other end of the bridge.
31. A robotic surgical instrument according to any of paragraphs 28 to 30, wherein the bridge is at least 10 times narrower than the distance between the two retaining structures.
32. A robotic surgical instrument according to any of paragraphs 28 to 31, wherein the bridge is at least 10 times narrower than the second body part perpendicular to the direction of the linear motion of the transmission structure.
33. A robotic surgical instrument according to any of paragraphs 21 to 32, wherein the articulated end effector comprises two jaws, the shaft being configured to transfer drive to open or close the two jaws.
34. A robotic surgical instrument according to paragraph 33, wherein the transmission structure is configured to move linearly away from the shaft to transfer drive to the articulated end effector via the shaft so as to close the two jaws together, and move linearly towards the shaft to transfer drive to the articulated end effector via the shaft so as to open the jaws apart.
35. A robotic surgical instrument according to paragraph 34, wherein the two instrument interface elements are mechanically constrained to move linearly together away from the shaft to transfer drive to the articulated end effector via the shaft so as to close the two jaws together, and move linearly towards the shaft to transfer drive to the articulated end effector via the shaft so as to open the jaws apart.
36. A robotic surgical instrument according to paragraphs 33 to 35, wherein the shaft comprises an inner shaft and an outer shaft, the inner and outer shafts being concentric, wherein the outer shaft is rotatable in one rotation direction to transfer drive to open the two jaws, and rotatable in the opposing rotation direction to transfer drive to close the two jaws.
37. A robotic surgical instrument according to paragraph 23 or any of paragraphs 24 to 36 when dependent on paragraph 23, wherein the transmission structure comprises:
   a planar interfacing portion comprising the two retaining structures;
   a drive portion proximal to the shaft; and
   a connector portion connecting the planar interfacing portion to the drive portion;
   wherein the longitudinal axis of the drive portion is parallel to but offset from the longitudinal axis of the planar interfacing portion.
38. A robotic surgical instrument according to any of paragraphs 21 to 37, wherein the transmission structure comprises a slider projection exposed on the exterior of the robotic surgical instrument so as to enable a user to manually push the transmission structure linearly.
39. A robotic surgical instrument according to paragraph 38, wherein the slider projection comprises a textured surface.
40. A robotic surgical instrument according to any of paragraphs 21 to 39, wherein the robotic surgical instrument is an ultrasonic surgical instrument.

1. A robotic surgical instrument comprising:
an articulated end effector;
an instrument interface configured to engage with and be driven by a corresponding robot arm interface of a surgical robot arm;
a casing housing a drive mechanism connected to the instrument interface, the drive mechanism comprising a drive gear; and
a shaft pivotally connected to the casing at a proximal end and connected to the articulated end effector at a distal end, the shaft comprising a driveable shaft member configured to drive articulation of the articulated end effector, and a shaft gear attached to the driveable shaft member, the shaft gear meshing with the drive gear such that rotation of the drive gear drives the shaft gear to rotate which drives the driveable shaft member which drives articulation of the articulated end effector, wherein the shaft gear is pivotable with respect to the drive gear when the shaft gear and drive gear are meshed together.

2. A robotic surgical instrument as claimed in claim 1, wherein the teeth of the shaft gear have rounded ends such that when meshed with the teeth of the drive gear, each tooth of the shaft gear extends only partway down the valley between adjacent teeth of the drive gear.

3. A robotic surgical instrument as claimed in claim 1, wherein the teeth of the drive gear have rounded ends such that when meshed with the teeth of the shaft gear, each tooth of the drive gear extends only partway down the valley between adjacent teeth of the shaft gear.

4. A robotic surgical instrument as claimed in claim 1, wherein the shaft comprises a protrusion rigidly attached to the shaft, wherein the protrusion extends in a direction perpendicular to the longitudinal axis of the driveable shaft member.

5. A robotic surgical instrument as claimed in claim 4, wherein the shaft gear comprises a ring surrounding the driveable shaft member, the teeth of the shaft gear extending from the ring, and the protrusion extending from the ring in a direction opposing the teeth of the shaft gear.

6. A robotic surgical instrument as claimed in claim 4, wherein the casing comprises a notch which constrains the protrusion when the longitudinal axis of the shaft is angled relative to the longitudinal axis of the instrument interface.

7. A robotic surgical instrument as claimed in claim 6, wherein the articulated end effector comprises a pair of opposable end effector elements, the opening angle between the end effector elements driveable by the driveable shaft member, wherein when the protrusion is constrained within the notch, the end effector elements are in an open configuration.

8. A robotic surgical instrument as claimed in claim 7, wherein when the protrusion is constrained within the notch, the opening angle between the end effector elements is greater than 200.

9. A robotic surgical instrument as claimed in claim 1, wherein the drive gear is a part gear.

10. A robotic surgical instrument as claimed in claim 9, wherein the teeth of the drive gear extend less than 90° around its centre.

11. A robotic surgical instrument as claimed in claim 1, wherein the shaft gear is a part gear.

12. A robotic surgical instrument as claimed in claim 11, wherein the teeth of the shaft gear extend less than 90° around the driveable shaft member.

13. A robotic surgical instrument as claimed in claim 1, wherein the driveable shaft member is a rotatable shaft member, and the drive mechanism comprises:
a transmission structure configured to transfer drive by moving linearly; and
a drive assembly configured to convert linear motion of the transmission structure to rotational motion in order to drive the rotatable shaft member, the drive assembly comprising a helical drive driveable by the transmission structure, the drive gear rigidly attached to the helical drive.

14. A robotic surgical instrument as claimed in claim 13, wherein the longitudinal axis of the helical drive is offset from the longitudinal axis of the rotatable shaft member.

15. A robotic surgical instrument as claimed in claim 13, wherein the longitudinal axis of the helical drive is parallel to the longitudinal axis of the rotatable shaft member.

16. A robotic surgical instrument as claimed in claim 15, wherein the helical drive, rotatable shaft member, drive gear and shaft gear are located in the same plane perpendicular to the longitudinal axes of the helical drive and rotatable shaft member.

17. A robotic surgical instrument as claimed in claim 13, wherein the teeth of the drive gear extend in a direction perpendicular to the longitudinal axis of the helical drive.

18. A robotic surgical instrument as claimed in claim 1, wherein the teeth of the shaft gear extend in a direction perpendicular to the longitudinal axis of the driveable shaft member.

19. A robotic surgical instrument as claimed in claim 13, wherein the teeth of the drive gear mesh with the teeth of the shaft gear in a direction perpendicular to the longitudinal axis of the helical drive.

20. A robotic surgical instrument as claimed in claim 13, wherein the drive gear meshes with the shaft gear such that rotation of the helical drive in one rotational direction is converted to rotation of the rotatable shaft member in the opposing rotational direction.

21. A method of assembling a robotic surgical instrument comprising a first portion and a second portion, the first portion comprising a rod connected to a first end effector element at a distal end and an instrument body at a proximal end, the second portion comprising an instrument interface configured to engage with and be driven by a corresponding robot arm interface of a surgical robot arm, a casing housing a drive mechanism connecting the instrument interface to a shaft, the shaft connecting the drive mechanism to a second end effector element, the shaft comprising a driveable shaft member configured to drive articulation of the second end effector element, a protrusion attached to the driveable shaft member and extending in a direction perpendicular to the longitudinal axis of the driveable shaft member, the protrusion retainable in a notch in the casing, the method comprising:
inserting the rod of the first portion into the driveable shaft member of the second portion;
rotating the rod so as to rotate the driveable shaft member until the protrusion is aligned with the notch of the casing and the second end effector element adopts an open configuration;
pivoting the driveable shaft member relative to the casing such that the longitudinal axis of the driveable shaft member is angled relative to the longitudinal axis of the instrument interface so as to cause the protrusion to be retained in the notch;
fully inserting the rod into the driveable shaft member; and
pivoting the driveable shaft member relative to the casing such that the longitudinal axis of the driveable shaft member is parallel to the longitudinal axis of the instrument interface and the instrument body is retained in the casing of the second portion.
